SEQUENCE LISTING (10 sequences)

SEQ ID NO: 1 - protein, 117 aa, synthetic construct [sequence omitted]

SEQ ID NO: 2 - protein, 107 aa, synthetic construct [sequence omitted]

SEQ ID NO: 3 - protein, 8 aa, synthetic construct
  REGION 1..8: note = R2810 HCDR1
GFTFSNFG

SEQ ID NO: 4 - protein, 8 aa, synthetic construct
  REGION 1..8: note = R2810 HCDR2
ISGGGRDT

SEQ ID NO: 5 - protein, 10 aa, synthetic construct
  REGION 1..10: note = R2810 HCDR3
VKWGNIYFDY

SEQ ID NO: 6 - protein, 6 aa, synthetic construct
  REGION 1..6: note = R2810 LCDR1
LSINTF

SEQ ID NO: 7 - skipped

SEQ ID NO: 8 - protein, 9 aa, synthetic construct
  REGION 1..9: note = R2810 LCDR3
QQSSNTPFT

SEQ ID NO: 9 - protein, 444 aa, synthetic construct
  REGION 1..444: note = R2810 HC
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = R2810 LC
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0327313 A1
(43) Pub. Date: Oct. 22, 2026

(54) Administration of PD-1 Inhibitors for Treating Skin Cancer

(71) Applicant: Regeneron Pharmaceuticals, Inc., Tarrytown, NY (US)

(72) Inventors: Matthew G. Fury, New York, NY (US); Israel Lowy, Dobbs Ferry, NY (US)

(21) Appl. No.: 19/728,813

(22) Filed: Jul. 2, 2026

Related U.S. Application Data
(63) Continuation of application No. 17/433,003, filed on Feb. 27, 2020.
(60) Provisional application No. 62/811,827, filed on Feb. 28, 2019.

(51) Int. Cl. C07K 16/28; A61K 39/00; A61K 39/39; A61P 35/00

(52) U.S. Cl. CPC C07K 16/2818; A61K 39/00; A61K 39/39; A61P 35/00; A61K 2039/505; A61K 2039/545; A61K 2039/55

(57) ABSTRACT

The disclosure relates to methods for treating or inhibiting the growth of a tumor in a patient with a skin cancer, including administering to the patient a therapeutically effective amount of a programmed death 1 (PD-1) inhibitor (e.g., an antibody or antigen-binding fragment thereof that specifically binds PD-1, PD-L1, and/or PD-L2). In certain embodiments, the method includes administering a PD-1 inhibitor as adjuvant treatment after the patient has completed surgery and optionally radiation therapy for skin cancer, such as CSCC, and is at high risk for disease recurrence. In certain embodiments, the method includes administering a PD-1 inhibitor as neoadjuvant treatment before planned surgery for skin cancer. In certain embodiments, the method includes administering a PD-1 inhibitor as neoadjuvant treatment before planned surgery for skin cancer and subsequently administering to the patient a PD-1 inhibitor as adjuvant therapy after such surgery.

### CROSS-REFERENCE TO RELATED APPLICATIONS

This application is continuation of U.S. patent application Ser. No. 17/433,003 filed Aug. 23, 2021, which is a U.S. national phase application under 35 U.S.C. § 371 of International Patent Application No. PCT/US2020/020018 filed Feb. 27, 2020, which claims the benefit of priority to U.S. Provisional Patent Application No. 62/811,827 filed Feb. 28, 2019, the disclosures of which are hereby incorporated by reference herein in their entireties.

### SEQUENCE LISTING

The sequence listing of the present application is submitted electronically in xml format with a file name of “10707SeqList.xml,” creation date of Jun. 29, 2026, and a size of 13,170 bytes. The submitted sequence listing is part of the specification and is hereby incorporated by reference in its entirety

### FIELD OF THE INVENTION

The present disclosure relates to methods for treating or inhibiting the growth of a tumor that includes selecting a patient with skin cancer and administering to the patient a therapeutically effective amount of a programmed death 1 (PD-1) inhibitor.

### BACKGROUND

Skin cancer is the most common cancer in the United States (Guy et al., Am. J. Prev. Med. 48:183-87, 2015). An estimated 5.4 million cases of non-melanoma skin cancer, including basal cell carcinoma and squamous cell carcinoma, were diagnosed in the United States in 2012 (Rogers et al., JAMA Dermatol., 151(10):1081-86, 2015). Cutaneous squamous cell carcinoma (CSCC) is the second-most common malignancy in the United States, after basal cell carcinoma (BCC) (Karia et al., J. Am. Acad. Dermatol. 68:957-66, 2013). Chronic sun exposure is the dominant risk factor for non-melanoma skin cancers.

CSCC is a malignant proliferation of epidermal keratinocytes with invasion of the dermis and is distinguished from non-invasive precursor lesions such as actinic keratoses (Fernandez et al., Immunol Allergy Clin North Am 37(2):315-27, 2017). Worldwide incidence varies widely, with the highest incidence in Australia and the lowest incidence in parts of Africa (Lomas et al., Br J Dermatol, 166(5):1069-80, 2012). The precise incidence of CSCC is not known because it is not included in most cancer registries. However, the incidence of CSCC has increased in recent decades according to estimates that do not include patients with only non-invasive precursor lesions (Lomas et al., Br J Dermatol, 166(5):1069-80, 2012) (Que et al., J Am Acad Dermatol, 78(2):237-47, 2018) (Rogers et al., Arch Dermatol, 146(3):283-87, 2010).

Risk factors for CSCC include UV exposure, advanced age, and immunosuppression (Alam et al., New Engl. J. Med. 344:975-83, 2001; Madan, Lancet 375:673-85, 2010). Although the vast majority of individuals diagnosed with CSCC or BCC have a very favorable prognosis, CSCC has a greater propensity for aggressive recurrences than BCC. Also, individuals diagnosed with CSCC, unlike those diagnosed with BCC, have an increased mortality compared with age-matched controls (Rees et al., Int. J. Cancer 137:878-84, 2015).

Surgical resection is the centerpiece of clinical management of CSCC or BCC. The primary goal is complete resection of cancer, and acceptable cosmetic outcome is a secondary goal. Factors associated with poor prognosis in CSCC include tumor size>2 cm, tumor depth>2 mm, perineural invasion, host immunosuppression, and recurrent lesions. However, some patients who develop advanced CSCC, which encompasses both locally advanced and metastatic CSCC, are not candidates for surgery. Some such patients may be administered post-operative radiation therapy or chemotherapy, but these may not be attractive options due to safety and tolerability concerns.

Field cancerization, defined as multiple cancerous lesions in UV exposed sites, is a characteristic of many CSCC patients. Additionally, recurrent CSCC increases the risk of subsequent recurrences. In a single institution retrospective study of 212 patients, recurrent CSCCs were twice as likely to recur again after excisional surgery as compared to primary CSCCs (Harris et al., Otolaryngol Head Neck Surg, 156(5):863-69, 2017). Multiple surgeries over time can be disfiguring and lead to surgical fatigue—i.e., physical and emotional debilitation resulting from serial surgical procedures. Also, CSCC in subsites of the head and neck, such as ear, temple, and lip, have been associated with worse clinical outcomes (Brantsch et al., Lancet Oncol 9(8):713-20, 2008; Harris et al., Otolaryngol Head Neck Surg, 156(5):863-69, 2017; Thompson et al., JAMA Dermatol 2016; 152(4):419-28, 2016).

The most common clinical subtype of BCC is nodular BCC. Less common clinical subtypes are superficial, morphoeic (fibrosing), and fibroepithelial. Most patients are cured by surgery, but a small percentage of patients experience recurrent lesions or develop unresectable locally advanced or metastatic disease. Recognition of the oncogenic role of the G-protein receptor Smoothened (SMO) in BCC led to the development of vismodegib and sonidegib, orally available inhibitors of SMO, generally referred to as Hedgehog Inhibitors (HHIs). In addition to adverse side-effects of the HHIs, it was found that for patients that progress on one HHI (vismodegib), subsequent treatment with another HHI (sonedegib) did not result in tumor inhibition (Danial et al., Clin. Cancer Res. 22:1325-29, 2016).

Therefore, there remains a need to provide safe and effective therapies for skin cancer, such as CSCC and BCC, especially skin cancer that has recurred despite prior surgeries.

Further, a small fraction of CSCC patients are considered to have high risk CSCC, as assessed using a number of factors, including cancer staging using the American Joint Committee on Cancer, 8th Edition (AJCC, 2017), immune status, lymphovascular invasion, extent of nodal involvement, presence of extracapsular extension and treatment history. Post-operative radiotherapy is recommended in high risk cases (Bichakjian et al., J Natl Compr Canc Netw, 16(6):742-74, 2018) (Stratigos, Eur J Cancer, 51(14):1989-2007, 2015). However, high risk patients may relapse with locoregional recurrence or distant metastases (Porceddu et al., J Clin Oncol, 36(13):1275-83, 2018). Thus, there is an unmet need to reduce the risk of CSCC recurrence, especially in high risk patients.

### SUMMARY

In one aspect, the disclosed technology relates to a method of treating or inhibiting the growth of a tumor, including: (a) selecting a patient with a skin cancer, wherein the patient has completed surgery and/or radiation therapy to treat the skin cancer; and (b) subsequently administering to the patient an adjuvant treatment including a therapeutically effective amount of a programmed death 1 (PD-1) inhibitor. In one embodiment, the method of treating or inhibiting the growth of a tumor includes: (a) selecting a patient with a skin cancer, wherein the patient has completed surgery and optionally, post-surgery radiation therapy to treat the skin cancer; and (b) subsequently administering to the patient an adjuvant treatment including a therapeutically effective amount of a programmed death 1 (PD-1) inhibitor. In one embodiment, the skin cancer is cutaneous squamous cell carcinoma (CSCC), basal cell carcinoma (BCC), Merkel cell carcinoma, or melanoma. In another embodiment, the skin cancer is CSCC. In another embodiment, the patient is at high risk of CSCC recurrence. In one embodiment, the patient has metastatic disease and has undergone respective surgery. In another embodiment, the patient has at least one of the following high-risk features: nodal disease with extracapsular extension and at least 1 node>20 mm; in-transit metastases (ITM); T4 lesion; perineural invasion (PNI); and recurrent CSCC plus at least one of the following additional features: ≥N2b disease associated with a recurrent lesion; nominal≥T3; and ≥20 mm diameter of recurrent lesion.

In another embodiment, the therapeutically effective amount includes 5 mg to 500 mg of the PD-1 inhibitor. In another embodiment, the therapeutically effective amount includes 350 mg of the PD-1 inhibitor. In another embodiment, the PD-1 inhibitor is administered intravenously or intraperitoneally. In another embodiment, the PD-1 inhibitor is administered intravenously. In another embodiment, step (b) occurs 2 to 6 weeks after completion of the surgery and/or radiation therapy. In one embodiment, one or more doses of the PD-1 inhibitor are administered as an adjuvant treatment, wherein each dose is administered 2 to 12 weeks after the immediately preceding dose. In another embodiment, the PD-1 inhibitor is administered as an adjuvant treatment according to an administration regimen as disclosed herein. In another embodiment, administration of the PD-1 inhibitor leads to reduced risk of subsequent skin cancer recurrence or zero incidence of subsequent skin cancer recurrence. In another embodiment, administration of the PD-1 inhibitor leads to at least about 10% lower incidence of subsequent skin cancer recurrence as compared to a patient after completion of surgery and radiation therapy without adjuvant skin cancer treatment. In another embodiment, the method further includes administering a second therapeutic agent selected from a chemotherapeutic, a corticosteroid, an anti-inflammatory drug, and/or combinations thereof.

In another embodiment, the PD-1 inhibitor is selected from the group consisting of an anti-PD-1 antibody or antigen-binding fragment thereof, an anti-PD-L1 antibody or antigen-binding fragment thereof, and an anti-PD-L2 antibody or antigen-binding fragment thereof. In another embodiment, the PD-1 inhibitor is an anti-PD-1 antibody or antigen-binding fragment thereof that includes three complementarity determining regions (CDRs) (HCDR1, HCDR2, and HCDR3) of a heavy chain variable region (HCVR) including the amino acid sequence of SEQ ID NO: 1 and three CDRs (LCDR1, LCDR2, and LCDR3) of a light chain variable region (LCVR) including the amino acid sequence of SEQ ID NO: 2. In another embodiment, HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8. In another embodiment, the anti-PD-1 antibody or antigen-binding fragment thereof includes a HCVR/LCVR sequence pair of SEQ ID NOs: 1/2. In another embodiment, the anti-PD-1 antibody includes a heavy chain and a light chain, wherein the heavy chain has an amino acid sequence of SEQ ID NO: 9. In another embodiment, the anti-PD-1 antibody includes a heavy chain and a light chain, wherein the light chain has an amino acid sequence of SEQ ID NO: 10. In another embodiment, the anti-PD-1 antibody includes a heavy chain and a light chain, wherein the heavy chain has an amino acid sequence of SEQ ID NO: 9 and the light chain has an amino acid sequence of SEQ ID NO: 10.

In another embodiment, the PD-1 inhibitor is cemiplimab or a bioequivalent thereof. In another embodiment, the PD-1 inhibitor is an anti-PD-1 antibody selected from the group consisting of cemiplimab, nivolumab, pembrolizumab, pidilizumab, MED10608, BI 754091, PF-06801591, spartalizumab, camrelizumab, JNJ-63723283, and MCLA-134. In another embodiment, the PD-1 inhibitor is an anti-PD-L1 antibody selected from the group consisting of H1H8314N, avelumab, atezolizumab, durvalumab, MDX-1105, LY3300054, FAZ053, STI-1014, CX-072, KN035, and CK-301.

In another aspect, the disclosed technology relates to a pharmaceutical composition including a therapeutically effective amount of a programmed death 1 (PD-1) inhibitor for use in an adjuvant treatment of skin cancer after completion of surgery and optionally, post-surgery radiation. In one embodiment, the PD-1 inhibitor is an anti-PD-1 antibody or antigen-binding fragment thereof including three complementarity determining regions (CDRs) (HCDR1, HCDR2, and HCDR3) of a heavy chain variable region (HCVR) including the amino acid sequence of SEQ ID NO: 1 and three CDRs (LCDR1, LCDR2, and LCDR3) of a light chain variable region (LCVR) including the amino acid sequence of SEQ ID NO: 2. In another embodiment, HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8. In another embodiment, the anti-PD-1 antibody or antigen-binding fragment thereof includes a HCVR/LCVR sequence pair of SEQ ID NOs: 1/2. In another embodiment, the pharmaceutical composition includes 5 mg to 500 mg of the PD-1 inhibitor. In another embodiment, the pharmaceutical composition includes 350 mg of the PD-1 inhibitor. In another embodiment, the skin cancer is CSCC.

In another aspect, the disclosed technology relates to a method of treating or inhibiting the growth of a tumor, including: (a) selecting a patient with a skin cancer for which surgical removal is planned; and (b) prior to the surgical removal, administering to the patient a neoadjuvant treatment including a therapeutically effective amount of a programmed death 1 (PD-1) inhibitor. In one embodiment, the skin cancer is cutaneous squamous cell carcinoma (CSCC), basal cell carcinoma (BCC), Merkel cell carcinoma, or melanoma. In another embodiment, the skin cancer is CSCC. In another embodiment, the patient is at high risk of CSCC recurrence. In another embodiment, the patient has at least one of the following high risk features: nodal disease with extracapsular extension and at least 1 node>20 mm; in-transit metastases (ITM); T4 lesion; perineural invasion (PNI); and recurrent CSCC plus at least one of the following additional features: ≥N2b disease associated with a recurrent lesion; nominal≥T3; and ≥20 mm diameter of recurrent lesion. In another embodiment, the patient has stage II to stage IV skin cancer wherein the patient is a candidate for surgery. In one embodiment, the patient with resectable tumor has an increased risk of recurrence and/or risk of disfigurement or loss of function.

In another embodiment, the therapeutically effective amount includes 5 mg to 500 mg of the PD-1 inhibitor administered as a neoadjuvant. In another embodiment, the therapeutically effective amount includes 350 mg of the PD-1 inhibitor administered as the neoadjuvant. In one embodiment, one or more doses of the PD-1 inhibitor are administered as neoadjuvant treatment, wherein each dose is administered 2 to 12 weeks after the immediately preceding dose. In another embodiment, the method further includes: (c) subsequent to the neoadjuvant treatment, surgically removing the skin cancer. In another embodiment, the method further includes administering to the patient an adjuvant treatment including a therapeutically effective amount of a PD-1 inhibitor after step (c), wherein the adjuvant PD-1 inhibitor may be the same as or different from the neoadjuvant PD-1 inhibitor. In another embodiment, the adjuvant treatment includes administering one or more doses of the PD-1 inhibitor, wherein each dose includes 5 mg to 500 mg of the PD-1 inhibitor. In another embodiment, each dose of the adjuvant treatment includes 350 mg of the PD-1 inhibitor. In one embodiment, each dose of the adjuvant treatment is administered 2 to 12 weeks after the immediately preceding dose. In another embodiment, the PD-1 inhibitor is administered intravenously or intraperitoneally. In another embodiment, the PD-1 inhibitor is administered intravenously.

In another embodiment, administration of the PD-1 inhibitor leads to reduced risk of subsequent skin cancer recurrence or zero incidence of subsequent skin cancer recurrence. In another embodiment, administration of the PD-1 inhibitor leads to at least about 10% lower incidence of subsequent skin cancer recurrence as compared to a patient after completion of surgery and radiation therapy without adjuvant skin cancer treatment. In another embodiment, the method further includes administering a second therapeutic agent selected from a chemotherapeutic, a corticosteroid, an anti-inflammatory drug, and/or combinations thereof. In another embodiment, the PD-1 inhibitor is selected from the group consisting of an anti-PD-1 antibody or antigen-binding fragment thereof, an anti-PD-L1 antibody or antigen-binding fragment thereof, and an anti-PD-L2 antibody or antigen-binding fragment thereof.

In another embodiment, the PD-1 inhibitor is an anti-PD-1 antibody or antigen-binding fragment thereof that includes three complementarity determining regions (CDRs) (HCDR1, HCDR2, and HCDR3) of a heavy chain variable region (HCVR) including the amino acid sequence of SEQ ID NO: 1 and three CDRs (LCDR1, LCDR2, and LCDR3) of a light chain variable region (LCVR) including the amino acid sequence of SEQ ID NO: 2. In another embodiment, HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8. In another embodiment, the anti-PD-1 antibody or antigen-binding fragment thereof includes a HCVR/LCVR sequence pair of SEQ ID NOs: 1/2. In another embodiment, the anti-PD-1 antibody includes a heavy chain and a light chain, wherein the heavy chain has an amino acid sequence of SEQ ID NO: 9. In another embodiment, the anti-PD-1 antibody includes a heavy chain and a light chain, wherein the light chain has an amino acid sequence of SEQ ID NO: 10. In another embodiment, the anti-PD-1 antibody includes a heavy chain and a light chain, wherein the heavy chain has an amino acid sequence of SEQ ID NO: 9 and the light chain has an amino acid sequence of SEQ ID NO: 10.

In another embodiment, the PD-1 inhibitor is cemiplimab or a bioequivalent thereof. In another embodiment, the PD-1 inhibitor is an anti-PD-1 antibody selected from the group consisting of cemiplimab, nivolumab, pembrolizumab, pidilizumab, MED10608, BI 754048, PF-06371548, spartalizumab, camrelizumab, JNJ-63313240, and MCLA-134. In another embodiment, the PD-1 inhibitor is an anti-PD-L1 antibody selected from the group consisting of H1H8314N, avelumab, atezolizumab, durvalumab, MDX-1105, LY3300054, FAZ053, STI-1014, CX-031, KN035, and CK-301.

In another aspect, the disclosed technology relates to a pharmaceutical composition including a therapeutically effective amount of a programmed death 1 (PD-1) inhibitor for use in a neoadjuvant treatment prior to planned surgery for treating skin cancer. In one embodiment, the PD-1 inhibitor is an anti-PD-1 antibody or antigen-binding fragment thereof including three complementarity determining regions (CDRs) (HCDR1, HCDR2, and HCDR3) of a heavy chain variable region (HCVR) including the amino acid sequence of SEQ ID NO: 1 and three CDRs (LCDR1, LCDR2, and LCDR3) of a light chain variable region (LCVR) including the amino acid sequence of SEQ ID NO: 2. In another embodiment, HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8. In another embodiment, the anti-PD-1 antibody or antigen-binding fragment thereof includes a HCVR/LCVR sequence pair of SEQ ID NOs: 1/2. In another embodiment, the pharmaceutical composition includes 5 mg to 500 mg of the PD-1 inhibitor. In another embodiment, the pharmaceutical composition includes 350 mg of the PD-1 inhibitor. In another embodiment, the skin cancer is CSCC.

As used herein, “the PD-1 inhibitor” may refer to at least one of the neadjuvant PD-1 inhibitor and the adjuvant PD-1 inhibitor.

### BRIEF DESCRIPTION OF THE DRAWINGS

FIG. 1 shows a diagram outlining the study described in Example 1.

FIG. 2 shows a diagram outlining the study described in Example 2.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims.

Unless defined otherwise, all technical and scientific terms used herein have the same meaning as commonly understood by one of ordinary skill in the art to which the disclosed invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, the preferred methods and materials are now described.

### Methods of Treating or Inhibiting Growth of a Tumor

The present disclosure includes methods for treating or inhibiting the growth of skin cancer comprising selecting a patient with a skin cancer and administering to the patient a therapeutically effective amount of a PD-1 inhibitor (e.g., an antibody or antigen-binding fragment thereof that specifically binds PD-1, PD-L1, and/or PD-L2, or any other “PD-1 inhibitor” as described herein). In the present disclosure, references to anti-PD-1 antibodies in particular are provided to illustrate a representative PD-1 inhibitor, and do not limit the scope of the disclosure. In one embodiment, the PD-1 inhibitor is administered prior to treating the patient with surgery and/or radiation therapy. In certain embodiments, the methods include administering to a subject in need thereof a PD-1 inhibitor as an adjuvant treatment after completion of surgery and optionally radiation therapy, such as post-operative radiation therapy, for treating skin cancer. In certain embodiments, the methods include administering to a subject in need thereof a PD-1 inhibitor as a neoadjuvant treatment prior to planned surgery for treating skin cancer, wherein the method may optionally further include subsequently administering to the patient a PD-1 inhibitor as adjuvant therapy after surgery for treating skin cancer.

In certain embodiments, present disclosure includes methods for treating or inhibiting the growth of skin cancer comprising (a) selecting a patient with a skin cancer, wherein the patient has at least one of the following high risk features: nodal disease with extracapsular extension and at least 1 node>20 mm; in-transit metastases (ITM); T4 lesion; perineural invasion (PNI); and recurrent CSCC plus at least one of the following additional features: ≥N2b disease associated with a recurrent lesion; nominal≥T3; and ≥20 mm diameter of recurrent lesion; and (b) administering to the patient in need thereof a therapeutically effective amount of a PD-1 inhibitor as adjuvant or neo-adjuvant treatment.

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In many embodiments, the terms “tumor”, “lesion,” “tumor lesion,” “cancer,” and “malignancy” are used interchangeably and refer to one or more growths.

In some embodiments, the skin cancer is cutaneous squamous cell carcinoma (CSCC), basal cell carcinoma (BCC), Merkel cell carcinoma, or melanoma. In some embodiments, the skin cancer is a squamous cell carcinoma of head and neck. In some embodiments, the skin cancer is advanced CSCC. In some embodiments, the skin cancer is a metastatic, resectable, unresectable, recurrent, or locally advanced. In some embodiments, the skin cancer is CSCC, including but not limited to metastatic CSCC, locally advanced CSCC, resectable CSCC, unresectable CSCC, or recurrent CSCC. In one embodiment, the skin cancer is CSCC that is resectable and recurrent.

As used herein, the term “recurrent” refers to a frequent or repeated diagnosis of skin cancer (e.g., CSCC) in a patient or a frequent or repeated occurrence of individual tumor lesion(s), such as primary tumor lesions and/or new tumor lesions that may represent recurrence of a prior tumor lesion.

As used herein, the term “recurrence” is defined as the appearance of one or more new skin cancer (e.g., CSCC) lesions that are local, regional, or distant. In many instances, new lesions in the skin are new primary tumors due to field cancerization from chronic UV-mediated skin damage (Christensen, F1000Res, 7, 2018). With respect to CSCC, local or regional (locoregional) recurrence is defined by any of the following sites of disease recurrence: (a) for HN CSCC, nodal or soft tissue recurrence above the clavicle; (b) for non-HN CSCC, recurrence within the first draining nodal basin (or soft tissue associated within the first draining nodal basin) of the resected tumor; (c) in-transit metastases, defined as skin or subcutaneous metastases that are >2 cm from the primary lesion but are not beyond the regional nodal basin. Distance recurrence is defined by any of the following sites of disease recurrence: (a) for HN CSCC, nodal recurrence below the clavicle; (d) for non-HN CSCC, recurrence beyond the first draining nodal basin of the resected tumor bed. Recurrence in 2 nodal basins will be considered distant recurrence, even if contiguous (i.e., 2 mediastinal nodal basins, 2 pelvic nodal basins); (e) recurrence in non-nodal tissue (including, but not limited to, lung, liver, bone, brain); (f) epidermotropic metastases, defined as distant lesion(s) in the dermis without epidermal involvement.

As used herein, the expression “a subject in need thereof” or “a patient in need thereof” means a human or non-human mammal that exhibits one or more symptoms or indications of skin cancer, and/or who has been diagnosed with skin cancer, including a solid tumor and who needs treatment for the same. In many embodiments, the terms “subject” and “patient” are used interchangeably. The expression includes subjects with primary, established, or recurrent tumor lesions. In specific embodiments, the expression includes human subjects that have and/or need treatment for a solid tumor. The expression also includes subjects with primary or metastatic tumors (advanced malignancies). In certain embodiments, the expression includes patients with a solid tumor that is resistant to or refractory to or is inadequately controlled by prior therapy (e.g., surgery or treatment with an anti-cancer agent such as carboplatin or docetaxel). In certain embodiments, the expression includes patients with a tumor lesion that has been treated with one or more lines of prior therapy (e.g., surgically removed), but which has subsequently recurred. In certain embodiments, the expression includes subjects with a skin cancer tumor lesion who are not candidates for curative surgery or curative radiation, or for whom conventional anti-cancer therapy is inadvisable, for example, due to toxic side effects. In other embodiments, the expression includes subjects with a skin cancer tumor lesion for which surgical removal is planned. In other embodiments, the expression includes subjects for whom the risk of recurrence is high due to prior history of recurrence after surgery.

In certain embodiments, the methods of the present disclosure are used in a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas. In certain embodiments, the term “solid tumor” comprises more than one tumor lesions located separate from one another, e.g., 2 or more, 5 or more, 10 or more, 15 or more, 20 or more, 25 or more lesions in a subject in need of treatment. In certain embodiments, the more than one lesions are located distally from one another.

In certain embodiments, the disclosed methods include administering a therapeutically effective amount of a PD-1 inhibitor (e.g., an anti-PD-1 antibody) in combination with an anti-tumor therapy. As used here, the expression “in combination with” means that the PD-1 inhibitor is administered before, after, or concurrent with the anti-tumor therapy. Anti-tumor therapies include, but are not limited to, conventional anti-tumor therapies such as chemotherapy, radiation, surgery, or as elsewhere described herein. In one embodiment, the anti-tumor therapy comprises surgery. In one embodiment, the PD-1 inhibitor is administered as neo-adjuvant treatment before surgery and/or radiation therapy. In another embodiment, the PD-1 inhibitor (e.g., anti-PD-1 antibody) is administered as an adjuvant treatment after surgery, after radiation therapy, or after surgery and post-operative radiation therapy. In certain embodiments, the PD-1 inhibitor (e.g., anti-PD-1 antibody) is administered after surgery and optionally post-operative radiation therapy.

In certain embodiments, the methods of the present disclosure comprise: (a) selecting a patient with a skin cancer wherein the skin cancer is selected from CSCC, BCC, Merkel cell carcinoma, or melanoma, and wherein the patient has one of the following high risk features: nodal disease with extracapsular extension and at least 1 node>20 mm; in-transit metastases (ITM); T4 lesion; perineural invasion (PNI); and recurrent CSCC plus at least one of the following additional features: ≥N2b disease associated with a recurrent lesion; nominal≥T3; and ≥20 mm diameter of recurrent lesion; and (b) administering a therapeutically effective amount of a PD-1 inhibitor (e.g., an anti-PD-1 antibody) to the patient in need thereof. In some embodiments, the patient has an advanced solid tumor, such as CSCC. In certain embodiments, the advanced solid tumor is indolent or aggressive. In one embodiment, the patient has had prior surgery and/or radiation to treat the skin cancer. In some embodiments, methods of the present disclosure include selecting a CSCC patient, who is at high risk for recurrence of CSCC, on the basis of the patient exhibiting one of the following high risk features: nodal disease with extracapsular extension and at least 1 node>20 mm; in-transit metastases (ITM); T4 lesion; perineural invasion (PNI); and recurrent CSCC plus at least one of the following additional features: ≥N2b disease associated with a recurrent lesion; nominal≥T3; and ≥20 mm diameter of recurrent lesion, and optionally wherein the patient has undergone respective surgery.

In certain embodiments, the subject is not responsive to, or has relapsed (e.g., experienced a recurrent lesion) after, prior therapy or surgery. In certain embodiments, the PD-1 inhibitor (e.g., an anti-PD-1 antibody) is administered intravenously or intraperitoneally to the subject. In some embodiments, the subject has CSCC with high risk tumor features, such as high-risk nodal disease, T4 tumor, perineural invasion, in-transit metastases, or history of recurrence plus at least one other risk factor.

As used herein, the expression “high risk” with respect to recurrence of CSCC or disease recurrence in a CSCC patient refers to at least one of the following factors: (a) Nodal disease with extracapsular extension (ECE) and at least 1 node>20 mm on the surgical pathology report (ECE is defined as extension through the lymph node capsule into the surrounding connective tissue, with or without associated stromal reaction, including for example, invasion of skin and infiltration of musculature/fixation to adjacent structures on clinical examination; (b) In-transit metastases (ITM), defined as skin or subcutaneous metastases that are >2 cm from the primary lesion but are not beyond the regional nodal basin (Leitenberger et al., J Am Acad Dermatol, 75(5):1022-31, 2016); (c) T4 lesion, including HN lesions (AJCC, 2017) and non-HN lesions (UICC, 2015); (d) Perineural invasion (PNI), defined as clinical and/or radiologic involvement of named nerves (UICC, Manual of Clinical Oncology, 9th ed., 2015) (e) Recurrent CSCC, defined as CSCC that arises within the area of the previously resected tumor, plus at least one of the following additional features (AJCC, 2017): (i) ≥N2b disease associated with the recurrent lesion, (ii) Nominal≥T3 (recurrent lesion 4 cm in diameter or minor bone erosion or deep invasion>6 mm measured from the granular layer of normal adjacent epithelium), or (iii) Poorly differentiated histology and ≥20 mm diameter of recurrent lesion (the recurrent tumor must be documented to be within the area of the previously resected CSCC by radial measurement of the greatest radius of the final defect, measured from the estimated center of the original surgical wound).

The methods of the present disclosure, according to certain embodiments, include intravenously or intraperitoneally administering to a subject a therapeutically effective amount of a PD-1 inhibitor (e.g., an anti-PD-1 antibody) in combination with an additional therapeutic agent, therapeutic regimen, or therapeutic procedure. The additional therapeutic agent, therapeutic regimen, or therapeutic procedure may be administered for increasing anti-tumor efficacy, for reducing toxic effects of one or more therapies and/or for reducing the dosage of one or more therapies. In various embodiments, the additional therapeutic agent, therapeutic regimen, or therapeutic procedure may include one or more of: chemotherapy, cyclophosphamide, surgery, a cancer vaccine, a programmed death ligand 1 (PD-L1) inhibitor (e.g., an anti-PD-L1 antibody), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CA28 activator, a 4-1BB agonist, a CD38 inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, an anti-CD3 antibody, a transforming growth factor beta (TGF3) inhibitor, an epidermal growth factor receptor (EGFR) inhibitor, an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], an anti-CD3/anti-CD20 bispecific antibody, a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-21, and IL-15, an anti-inflammatory drug such as a corticosteroid, a non-steroidal anti-inflammatory drug (NSAID), and a dietary supplement such as an antioxidant. In certain embodiments, the PD-1 inhibitor (e.g., an anti-PD-1 antibody) may be administered in combination with therapy including a chemotherapeutic agent and/or surgery.

In certain embodiments, administering to a subject with skin cancer a PD-1 inhibitor (e.g., an anti-PD-1 antibody) as an adjuvant treatment after completion of surgery and optionally radiation therapy, such as post-operative radiation therapy, leads to complete disappearance of all evidence of tumor cells (“complete response”), leads to at least 30% or more decrease in tumor cells or tumor size (“partial response”), or leads to complete or partial disappearance of tumor cells/lesions including new measurable lesions. Tumor reduction can be measured by any methods known in the art, e.g., X-rays, positron emission tomography (PET), computed tomography (CT), magnetic resonance imaging (MRI), cytology, histology, or molecular genetic analyses.

In certain embodiments, administering to a subject with skin cancer a PD-1 inhibitor (e.g., an anti-PD-1 antibody) as an adjuvant treatment after completion of surgery and optionally radiation therapy, such as post-operative radiation therapy, leads to increased overall survival (OS) or progression-free survival (PFS) of the subject as compared to a subject administered with a ‘standard-of-care’ (SOC) therapy (e.g., chemotherapy, surgery or radiation). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies.

In certain embodiments, administration of a therapeutically effective amount of a PD-1 inhibitor (e.g., an anti-PD-1 antibody) to a subject with skin cancer as an adjuvant treatment after completion of surgery and optionally post-operative radiation therapy, wherein the subject is at high risk for recurrence, leads to a reduced risk of subsequent skin cancer recurrence or zero incidence of skin cancer recurrence. In certain embodiments, administration of a therapeutically effective amount of a PD-1 inhibitor (e.g., an anti-PD-1 antibody) to a CSCC patient after completion of surgery and/or radiation therapy for treating skin cancer leads to a reduced risk of subsequent CSCC recurrence or zero incidence of CSCC recurrence. In certain embodiments of the disclosed method, administration of a therapeutically effective amount of a PD-1 inhibitor (e.g., an anti-PD-1 antibody) to a CSCC patient after completion of surgery and radiation therapy for treating skin cancer leads to zero incidence of CSCC recurrence for at least 6 months or at least 1, 2, 3, 4, 5, 6, 7, 8, 9, or 10 years or longer after administration of the adjuvant PD-1 inhibitor.

In certain embodiments, administration of a therapeutically effective amount of a PD-1 inhibitor (e.g., an anti-PD-1 antibody) to a skin cancer patient after completion of surgery and radiation therapy for treating skin cancer leads to at least about 10%, about 20%, about 30%, about 40%, about 50%, about 60%, about 70%, about 80%, or about 90% lower incidence of subsequent CSCC recurrence as compared to a patient treated with surgery and radiation therapy without adjuvant skin cancer treatment.

In certain embodiments, administering to a subject with skin cancer a PD-1 inhibitor (e.g., an anti-PD-1 antibody) as a neoadjuvant treatment prior to planned surgery for treating skin cancer, and optionally subsequently administering to the patient a PD-1 inhibitor as adjuvant therapy after surgery for treating skin cancer, leads to complete disappearance of all evidence of tumor cells (“complete response”), leads to at least 30% or more decrease in tumor cells or tumor size (“partial response”), or leads to complete or partial disappearance of tumor cells/lesions including new measurable lesions. Tumor reduction can be measured by any methods known in the art, e.g., X-rays, positron emission tomography (PET), computed tomography (CT), magnetic resonance imaging (MRI), cytology, histology, or molecular genetic analyses.

In certain embodiments, administering to a subject with skin cancer a PD-1 inhibitor (e.g., an anti-PD-1 antibody) as a neoadjuvant treatment prior to planned surgery for treating skin cancer, and optionally subsequently administering to the patient a PD-1 inhibitor as adjuvant therapy after surgery for treating skin cancer, leads to increased overall survival (OS) or progression-free survival (PFS) of the subject as compared to a subject administered with a ‘standard-of-care’ (SOC) therapy (e.g., chemotherapy, surgery or radiation). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies.

In certain embodiments, administration of a therapeutically effective amount of a PD-1 inhibitor (e.g., an anti-PD-1 antibody) to a subject with skin cancer as a neoadjuvant treatment prior to planned surgery leads to a reduced risk of subsequent skin cancer recurrence or zero incidence of skin cancer recurrence. In certain embodiments, administration of a therapeutically effective amount of a PD-1 inhibitor (e.g., an anti-PD-1 antibody) to a CSCC patient prior to surgical removal of a skin cancer lesion for treating skin cancer leads to a reduced risk of subsequent CSCC recurrence or zero incidence of CSCC recurrence. In certain embodiments of the disclosed method, administration of a therapeutically effective amount of a PD-1 inhibitor (e.g., an anti-PD-1 antibody) to a CSCC patient as neoadjuvant treatment prior to planned surgery for treating skin cancer leads to zero incidence of CSCC recurrence for at least 6 months or at least 1, 2, 3, 4, 5, 6, 7, 8, 9, or 10 years or longer after the surgery.

In certain embodiments, administration of a therapeutically effective amount of a PD-1 inhibitor (e.g., an anti-PD-1 antibody) to a skin cancer patient as a neoadjuvant treatment prior to planned surgery for treating skin cancer leads to at least about 10%, about 20%, about 30%, about 40%, about 50%, about 60%, about 70%, about 80%, or about 90% lower incidence of subsequent CSCC recurrence as compared to a patient not treated with surgery without neoadjuvant skin cancer treatment.

### PD-1 Inhibitors

The methods disclosed herein include administering a therapeutically effective amount of a PD-1 inhibitor. As used herein, a “PD-1 inhibitor” refers to any molecule capable of inhibiting, blocking, abrogating or interfering with the activity or expression of PD-1. In some embodiments, the PD-1 inhibitor can be an antibody, a small molecule compound, a nucleic acid, a polypeptide, or a functional fragment or variant thereof. Non-limiting examples of suitable PD-1 inhibitor antibodies include anti-PD-1 antibodies and antigen-binding fragments thereof, anti-PD-L1 antibodies and antigen-binding fragments thereof, and anti-PD-L2 antibodies and antigen-binding fragments thereof. Other non-limiting examples of suitable PD-1 inhibitors include RNAi molecules such as anti-PD-1 RNAi molecules, anti-PD-L1 RNAi, and an anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 inhibitors are described in e.g., U.S. Pat. No. 9,308,236, U.S. Ser. No. 10/011,656, and US 20170290808, the portions of which that identify PD-1 inhibitors are hereby incorporated by reference.

As used herein, the term “antibody” refers to immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding portion” of an antibody, “antigen-binding fragment” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}—C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes [see e.g., Taylor et al. (1992) Nucl. Acids Res. 20:6287-6295] or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Anti-PD-1 Antibodies and Antigen-Binding Fragments Thereof

In some embodiments, PD-1 inhibitors used in the methods disclosed herein are antibodies or antigen-binding fragments thereof that specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments, the anti-PD-1 antibody, or antigen-binding fragment thereof comprises a heavy chain variable region (HCVR), light chain variable region (LCVR), and/or complementarity determining regions (CDRs) comprising the amino acid sequences of any of the anti-PD-1 antibodies set forth in U.S. Pat. No. 9,987,500, which is hereby incorporated by reference in its entirety. In certain exemplary embodiments, the anti-PD-1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of AAS; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a heavy chain comprising the amino acid sequence of SEQ ID NO: 9 and a light chain comprising the amino acid sequence of SEQ ID NO: 10 is the fully human anti-PD-1 antibody known as cemiplimab (also known as REGN2810, LIBTAYO®).

According to certain exemplary embodiments, the methods of the present disclosure comprise the use of REGN2810, or a bioequivalent thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., REGN2810) when administered at the same molar dose under similar experimental conditions, either single dose or multiple dose. In the context of the present disclosure, the term “bioequivalent” includes antigen-binding proteins that bind to PD-1 and do not have clinically meaningful differences with REGN2810 with respect to safety, purity and/or potency.

According to certain embodiments of the present disclosure, the anti-human PD-1, or antigen-binding fragment thereof, comprises a HCVR having 90%, 95%, 98% or 99% sequence identity to SEQ ID NO: 1.

According to certain embodiments of the present disclosure, the anti-human PD-1, or antigen-binding fragment thereof, comprises a LCVR having 90%, 95%, 98% or 99% sequence identity to SEQ ID NO: 2.

According to certain embodiments of the present disclosure, the anti-human PD-1, or antigen-binding fragment thereof, comprises a HCVR comprising an amino acid sequence of SEQ ID NO: 1 having no more than 5 amino acid substitutions. According to certain embodiments of the present disclosure, the anti-human PD-1, or antigen-binding fragment thereof, comprises a LCVR comprising an amino acid sequence of SEQ ID NO: 2 having no more than 2 amino acid substitutions.

Sequence identity may be measured by methods known in the art (e.g., GAP, BESTFIT, and BLAST).

The present disclosure also includes use of anti-PD-1 antibodies in methods to treat skin cancer, wherein the anti-PD-1 antibodies comprise variants of any of the HCVR, LCVR and/or CDR amino acid sequences disclosed herein having one or more conservative amino acid substitutions. For example, the present disclosure includes use of anti-PD-1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-1 antibodies that can be used in the context of the methods of the present disclosure include, e.g., the antibodies referred to and known in the art as nivolumab, pembrolizumab, MEDI0608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO2006/121168, WO2009/114335. The portions of all of the aforementioned publications that identify anti-PD-1 antibodies are hereby incorporated by reference.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Anti-PD-L1 Antibodies and Antigen-Binding Fragments Thereof

In some embodiments, PD-1 inhibitors used in the methods disclosed herein are antibodies or antigen-binding fragments thereof that specifically bind PD-L1. For example, an antibody that “specifically binds” PD-L1, as used in the context of the present disclosure, includes antibodies that bind PD-L1 or a portion thereof with a K_{D} of about 1×10⁻⁸ M or less (e.g., a smaller K_{D} denotes a tighter binding). A “high affinity” anti-PD-L1 antibody refers to those mAbs having a binding affinity to PD-L1, expressed as K_{D} of at least 10⁻⁸ M, preferably 10⁻⁹ M, more preferably 10⁻¹⁰ M, even more preferably 10⁻¹¹ M, even more preferably 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

According to certain exemplary embodiments, the anti-PD-L1 antibody, or antigen-binding fragment thereof comprises a heavy chain variable region (HCVR), light chain variable region (LCVR), and/or complementarity determining regions (CDRs) comprising the amino acid sequences of any of the anti-PD-L1 antibodies set forth in U.S. Pat. No. 9,938,345, which is hereby incorporated by reference in its entirety. In certain exemplary embodiments, an anti-PD-L1 antibody or antigen-binding fragment thereof that can be used in the context of the present disclosure comprises the heavy chain complementarity determining regions (HCDRs) of a heavy chain variable region (HCVR) and the light chain complementarity determining regions (LCDRs) of a light chain variable region (LCVR), wherein the HCVR and LCVR comprise the amino acid sequences of the anti-PD-L1 antibody designated as H1H8314N in U.S. Pat. No. 9,938,345. According to certain embodiments, the anti-PD-L1 antibody or antigen-binding fragment thereof comprises three HCDRs (HCDR1, HCDR2 and HCDR3) and three LCDRs (LCDR1, LCDR2 and LCDR3), wherein the HCDR1, HCDR2, HCDR3, LCDR1, LCDR2 and LCDR3 comprise the amino acid sequences of the anti-PD-L1 antibody designated as H1H8314N in U.S. Pat. No. 9,938,345. In yet other embodiments, the anti-PD-L1 antibody or antigen-binding fragment thereof comprises an HCVR and an LCVR that comprise the amino acid sequences of the anti-PD-L1 antibody designated as H1H8314N in U.S. Pat. No. 9,938,345.

According to certain embodiments of the present disclosure, the anti-human PD-L1, or antigen-binding fragment thereof, comprises a LCVR having 90%, 95%, 98% or 99% sequence identity to the LCVR amino acid sequence of the anti-PD-L1 antibody designated as H2M8314N in U.S. Pat. No. 9,938,345.

According to certain embodiments of the present disclosure, the anti-human PD-L1, or antigen-binding fragment thereof, comprises a HCVR comprising an amino acid sequence of the anti-PD-L1 antibody designated as H1H8314N in U.S. Pat. No. 9,938,345 having no more than 5 amino acid substitutions. According to certain embodiments of the present disclosure, the anti-human PD-L1, or antigen-binding fragment thereof, comprises a LCVR comprising an amino acid sequence of the anti-PD-L1 antibody designated as H1H8314N in U.S. Pat. No. 9,938,345 having no more than 2 amino acid substitutions.

Sequence identity may be measured by methods known in the art (e.g., GAP, BESTFIT, and BLAST).

The present disclosure also includes use of anti-PD-L1 antibodies in methods to treat skin cancer, wherein the anti-PD-L1 antibodies comprise variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-L1 antibodies that can be used in the context of the methods of the present disclosure include, e.g., the antibodies referred to and known in the art as MDX-1105, atezolizumab (TECENTRIQ™), durvalumab (IMFINZI™), avelumab (BAVENCIO™) LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016 Apr. 4 Abstract 4606), or any of the other anti-PD-L1 antibodies set forth in patent publications U.S. Pat. Nos. 7,943,743, 8,217,149, 9,402,899, 9,624,298, 9,938,345, WO 2007/005874, WO 2010/077634, WO 2013/181452, WO 2013/181634, WO 2016/149201, WO 2017/034916, or EP3177649. The portions of all of the aforementioned publications that identify anti-PD-L1 antibodies are hereby incorporated by reference.

### Pharmaceutical Compositions and Administration

The PD-1 inhibitors disclosed herein may be included within a pharmaceutical composition, which may be formulated with suitable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis. See, e.g., Wu et al., J. Biol. Chem. 262: 4429-32 (1987).

A pharmaceutical composition comprising a PD-1 inhibitor disclosed herein is suitable for intravenous administration, or administration intraperitoneally.

Injectable formulations of the pharmaceutical composition may be prepared by known methods. For example, the injectable formulation may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable formulation thus prepared is preferably filled in an appropriate injection ampoule. In some embodiments, an injectable formulation may be in the form of an injection solution that includes a concentration of PD-1 inhibitor and one or more solvents (e.g., distilled water, saline, etc.).

In certain embodiments, the present disclosure provides a pharmaceutical composition or formulation comprising a therapeutic amount of a PD-1 inhibitor (such as an anti-PD-1 antibody) and a pharmaceutically acceptable carrier. In certain embodiments, the present disclosure provides for a PD-1 inhibitor (such as an anti-PD-1 antibody) formulated in a pharmaceutical composition suitable for administration by intravenous injection.

Exemplary pharmaceutical compositions comprising an anti-PD-1 antibody that can be used in the context of the present disclosure are disclosed, e.g., in US 2019/0040137.

### Administration Regimens

In certain embodiments, the methods disclosed herein include administering to the tumor of a subject in need thereof a therapeutically effective amount of a PD-1 inhibitor (such as an anti-PD-1 antibody) in multiple doses, e.g., as part of a specific therapeutic dosing regimen. For example, a suitable therapeutic dosing regimen may comprise administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved. In one embodiment, one or more doses of an anti-PD-1 antibody are administered once a week.

In certain embodiments, the one or more doses are administered in at least one treatment cycle. The methods, according to this aspect, comprise administering to a subject in need thereof at least one treatment cycle comprising administration of 1, 2, 3, 4, 5, 6, 7, 8, 9, 10 or more doses of a PD-1 inhibitor (such as an anti-PD-1 antibody). In one embodiment, a treatment cycle comprises 3 doses of a PD-1 inhibitor. In one embodiment, a treatment cycle comprises 12 doses of a PD-1 inhibitor. In one embodiment, a treatment cycle comprises 24 doses of a PD-1 inhibitor. In one embodiment, a treatment cycle comprises 3 doses of the PD-1 inhibitor, each dose administered two weeks after the immediately preceding dose. In one embodiment, a treatment cycle comprises 10 doses of the PD-1 inhibitor, each dose administered one week after the immediately preceding dose. In one embodiment, a treatment cycle comprises 12 doses of the PD-1 inhibitor, each dose administered one week after the immediately preceding dose.

In one embodiment, all doses administered in a treatment cycle comprise the same amount of the PD-1 inhibitor. In another embodiment, a treatment cycle comprises administration of at least two doses that comprise different amounts of the PD-1 inhibitor. In one embodiment, the first dose in the treatment cycle comprises a larger amount of the PD-1 inhibitor than the subsequent doses in the treatment cycle. In one embodiment, the first dose in the treatment cycle comprises a smaller amount of the PD-1 inhibitor than the subsequent doses in the treatment cycle.

In one embodiment, all doses in a treatment cycle are administered using the same route of administration. In another embodiment, the doses in the treatment cycle are administered using different routes of administration, including two or more routes of administration.

In one embodiment, the treatment cycle is repeated. In some embodiments, the treatment cycle is repeated 1, 2, 3, 4, 5, 6, 7, 8, 9, 10, 11, 12, or more times.

In certain embodiments, a dose of a PD-1 inhibitor is administered to a subject in a single session or patient visit.

As used herein, the terms “initial,” “secondary,” “tertiary,” and so on refer to the temporal sequence of administration. Thus, an “initial dose” is a dose that is administered at the beginning of the treatment regimen (also referred to as a “baseline dose”); a “secondary dose” is a dose administered after the initial dose; and a “tertiary dose” is a dose administered after the secondary dose. The initial, secondary, and tertiary doses may all contain the same amount of the PD-1 inhibitor (anti-PD-1 antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an anti-PD-1 antibody may be administered to a patient with a cancer at a loading dose of about 1 mg/kg to about 3 mg/kg followed by one or more maintenance doses of about 0.1 mg/kg to about 20 mg/kg of the patient's body weight.

In one exemplary embodiment of the present disclosure, each secondary and/or tertiary dose is administered % to 4 weeks or more (e.g., %, 1, 1%, 2, 2%, 3, 3%, 4, or more weeks) after the immediately preceding dose. The phrase “the immediately preceding dose,” as used herein, means, in a sequence of multiple administrations, the dose of anti-PD-1 antibody administered to a subject prior to administration of the next dose in the sequence with no intervening doses.

Similarly, an “initial treatment cycle” is a treatment cycle that is administered at the beginning of the treatment regimen; a “secondary treatment cycle” is a treatment cycle administered after the initial treatment cycle; and a “tertiary treatment cycle” is a treatment cycle administered after the secondary treatment cycle. In the context of the present disclosure, treatment cycles may be the same or different from each other.

### Dosage

In certain embodiments, each dose of the PD-1 inhibitor comprises 0.1, 1, 0.3, 3, 4, 5, 6, 7, 8, 9 or 10 mg/kg of the patient's body weight. In certain embodiments, each dose comprises 5-500 mg of the PD-1 inhibitor, for example 5, 10, 15, 20, 25, 40, 45, 50, 60, 70, 80, 90, 100, 150, 200, 250, 300, 350, 400, 450 mg or more of the PD-1 inhibitor. In one embodiment, the PD-1 inhibitor is REGN2810 (cemiplimab).

The amount of PD-1 inhibitor administered to a subject (e.g., intravenously or intraperitoneally) according to the methods disclosed herein is, generally, a therapeutically effective amount. As used herein, the term “therapeutically effective amount” means an amount of a PD-1 inhibitor that results in one or more of: (a) a reduction in the severity or duration of a symptom or an indication of a skin cancer—e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; and/or (f) increase in survival of a subject with a cancer.

In the case of a PD-1 inhibitor (e.g., an anti-PD-1 antibody), a therapeutically effective amount can be from about 5 mg to about 500 mg, from about 10 mg to about 450 mg, from about 50 mg to about 400 mg, from about 75 mg to about 350 mg, or from about 100 mg to about 300 mg of the antibody. For example, in various embodiments, the amount of the PD-1 inhibitor is about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the PD-1 inhibitor.

In one embodiment, a therapeutically effective amount of 350 mg of a PD-1 inhibitor (e.g., an anti-PD-1 antibody) may be intravenously administered as an adjuvant treatment after surgery and optionally post-operative radiation therapy according to certain methods disclosed herein. In another embodiment, a therapeutically effective amount of 350 mg of a PD-1 inhibitor (e.g., an anti-PD-1 antibody) may be intravenously administered as a neoadjuvant treatment prior to planned surgery for treating skin cancer according to certain methods disclosed herein.

The amount of a PD-1 inhibitor contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure comprise administration of a PD-1 inhibitor (e.g., an anti-PD-1 antibody) at a dose of about 1 mg/kg, 3 mg/kg, 5 mg/kg or 10 mg/kg of a patient's body weight.

In certain embodiments, an individual dose amount of a PD-1 inhibitor (e.g., an anti-PD-1 antibody) administered to a patient (e.g., intravenously or intraperitoneally) may be less than a therapeutically effective amount, i.e., a subtherapeutic dose. For example, if the therapeutically effective amount of a PD-1 inhibitor comprises 3 mg/kg, a subtherapeutic dose comprises an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the PD-1 inhibitor that does not lead to a therapeutic effect by itself. However, in certain embodiments, multiple subtherapeutic doses of a PD-1 inhibitor are administered to collectively achieve a therapeutic effect in the subject.

In certain embodiments, each dose comprises 0.1-10 mg/kg (e.g., 0.3 mg/kg, 1 mg/kg, 3 mg/kg, or 10 mg/kg) of the subject's body weight. In certain other embodiments, each dose comprises 5-600 mg of the PD-1 inhibitor (such as an anti-PD-1 antibody), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, or 500 mg of the PD-1 inhibitor.

### EXAMPLES

The disclosed technology is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled. Also, while efforts have been made to ensure accuracy with respect to numbers used (e.g., amounts, temperature, etc.), some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Comparing Anti-PD-1 Antibody Versus Placebo as Adjuvant Treatment for CSCC Patients Post-Surgery and Post-Radiation Therapy

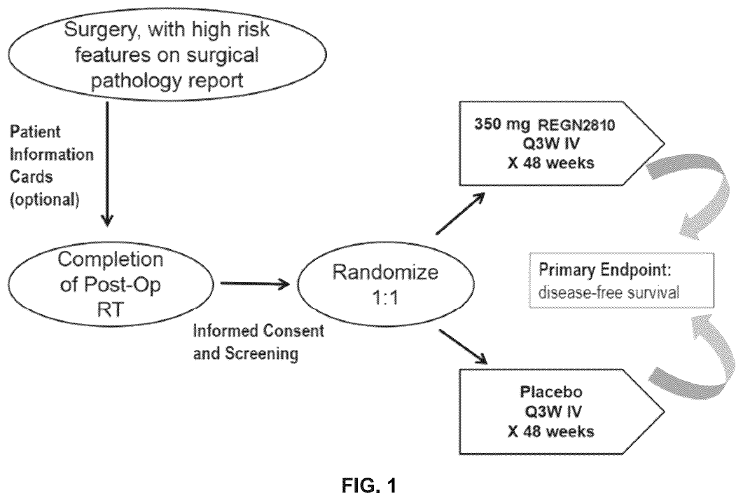

This study is a randomized, placebo-controlled, double-blind, multicenter, phase 3 study comparing an anti-PD-1 antibody, versus placebo, as adjuvant treatment for CSCC patients with features associated with high risk of recurrent disease, who have completed surgery and post-operative radiation therapy (RT). The study population comprises CSCC patients with high risk features on surgical pathology who have completed surgery and post-operative RT. See FIG. 1.

The exemplary anti-PD-1 antibody used in this study is REGN2810 (also known as cemiplimab, or H4H7798N as disclosed in U.S. Pat. No. 9,987,500), which is a fully human monoclonal anti-PD-1 antibody comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising SEQ ID NOs: 3-6, AAS, and SEQ ID NO: 8.

### Study Objectives

A primary objective of the study is to compare disease-free survival (DFS) of patients with high risk CSCC treated with adjuvant REGN2810, versus those treated with placebo, after surgery and RT.

Secondary objectives of the study include: (1) to compare the overall survival (OS) of high risk CSCC patients treated with adjuvant REGN2810, versus those treated with placebo, after surgery and RT; (2) to compare the effect of adjuvant REGN2810 with that of placebo on patients' freedom from locoregional recurrence (FFLRR) after surgery and RT; (3) to compare the effect of adjuvant REGN2810 with that of placebo on patients' freedom from distant recurrence (FFDR) after surgery and RT; (4) to compare the effect of adjuvant REGN2810 with that of placebo on the cumulative incidence of second primary CSCC tumors (SPTs) after surgery and RT; and (5) to evaluate the safety of adjuvant REGN2810 and that of placebo in high risk CSCC patients after surgery and RT.

### Study Duration

The duration of Part 1 of the study (blinded treatment period) is up to 48 weeks. The duration of Part 2 of the study (open-label REGN2810 treatment period) is up to 96 weeks.

### Study Population

Approximately 412 patients will be randomized into two treatment groups of approximately 206 patients each. The target patient population will consist of adult high risk CSCC patients who have undergone surgical resection followed by RT. Subject to the inclusion criteria below, Post-Operative Radiation Therapy (PORT) is delivered following complete macroscopic resection of high risk CSCC of head and neck (HN) and non-HN sites, prior to enrollment and randomization to the study.

Inclusion Criteria: A patient must meet the following criteria to be eligible for inclusion in the study: (1) at least 18 years old (for Japan only, at least 21 years old); (2) patient with resection of pathologically confirmed CSCC (primary CSCC lesion only, or primary CSCC with nodal involvement, or CSCC nodal metastasis with known primary CSCC lesion previously treated within the draining lymph node echelon), with macroscopic gross resection of all disease; (3) high risk CSCC, as defined by at least one of the following: (a) Nodal disease with extracapsular extension (ECE) and at least 1 node>20 mm on the surgical pathology report (ECE is defined as extension through the lymph node capsule into the surrounding connective tissue, with or without associated stromal reaction. Unambiguous evidence of gross ECE (defined as invasion of skin, infiltration of musculature/fixation to adjacent structures on clinical examination) is a sufficiently high threshold to classify these as ECE positive (AJCC, 2017)); (b) In-transit metastases (ITM), defined as skin or subcutaneous metastases that are >2 cm from the primary lesion but are not beyond the regional nodal basin (Leitenberger et al., J Am Acad Dermatol, 75(5):1022-31, 2016); (c) T4 lesion, including HN lesions (AJCC, 2017) and non-HN lesions (UICC, 2015); (d) Perineural invasion (PNI), defined as clinical and/or radiologic involvement of named nerves (UICC, Manual of Clinical Oncology, 9th ed., 2015) (e) Recurrent CSCC, defined as CSCC that arises within the area of the previously resected tumor, plus at least 1 of the following additional features (AJCC, 2017): (i) ≥N2b disease associated with the recurrent lesion, (ii) Nominal≥T3 (recurrent lesion 4 cm in diameter or minor bone erosion or deep invasion>6 mm measured from the granular layer of normal adjacent epithelium), or (iii) Poorly differentiated histology and ≥20 mm diameter of recurrent lesion (the recurrent tumor must be documented to be within the area of the previously resected CSCC by radial measurement of the greatest radius of the final defect, measured from the estimated center of the original surgical wound); (4) Completion of curative intent post-operative RT within 2 to 6 weeks of randomization. Patients must have received a minimum biologically Equivalent Dose in 2 Gy per fraction (EQD2) to the site of previous gross disease of 60 Gy for head and neck primary sites and 50 Gy for non-head and neck primary sites; (5) Eastern Cooperative Oncology Group performance status (ECOG PS)≤1; (6) Adequate hepatic function: a. Total bilirubin 51.5×upper limit of normal (ULN), b. Transaminases (aspartate aminotransferase [AST] and alanine aminotransferase [ALT])≤3×ULN, c. Alkaline phosphatase (ALP)≤2.5×ULN; (7) Adequate renal function: Serum creatinine≤1.5×ULN or estimated creatinine clearance (CrCl)>30 mL/min according to the method of Cockcroft and Gault; (8) Adequate bone marrow function: a. Hemoglobin≥9.0 g/dL, b. Absolute neutrophil count (ANC)≤1.0×109/L, c. Platelet count≥75×109/L; (9) Must be willing and able to provide informed consent signed by study patient or legally acceptable representative, as specified by health authorities and institutional guidelines; (10) All toxicities from radiotherapy must have resolved to grade 1 or less except dysgeusia, fatigue, xerostomia, trismus, alopecia, fibrosis, or edema in radiated field; (11) Willing and able to comply with clinic visits and study-related procedures; and (12) Able to understand and complete study-related questionnaires.

Exclusion Criteria: A patient who meets any of the following criteria will be excluded from the study: (1) Squamous cell carcinomas (SCCs) arising in non-cutaneous sites (e.g., dry red lip [vermillion], oral cavity, oropharynx, paranasal sinus, larynx, hypopharynx, nasopharynx, salivary gland, nasal mucosa, anogenital area, or SCC nodal metastasis with unknown primary); (2) Concurrent malignancy other than localized CSCC and/or history of malignancy other than localized CSCC within 3 years of date of randomization, except for tumors with negligible risk of metastasis or death, such as adequately treated (BCC) of the skin, carcinoma in situ of the cervix, or ductal carcinoma in situ of the breast, or low-risk early stage prostate adenocarcinoma (T1-T2ₐN0M0 and Gleason score 56 and prostate-specific antigen (PSA)≤10 ng/mL) for which the management plan is active surveillance, or prostate adenocarcinoma with biochemical-only recurrence with documented PSA doubling time of >12 months for which the management plan is active surveillance (D'Amico et al., JAMA, 294(4):440-47, 2005) (Pham et al., J Urol, 196(2):392-98, 2016); (3) Patients with hematologic malignancies (e.g., chronic lymphocytic leukemia [CLL]); (4) Patients with history of distantly metastatic CSCC (visceral or distant nodal), unless the disease-free interval is at least 3 years (regional nodal involvement of disease in draining lymph node basin that was resected and radiated prior to enrollment will not be exclusionary, per exclusion criterion 2); (5) Ongoing or recent (within 5 years of randomization date) evidence of significant autoimmune disease that required treatment with systemic immunosuppressive treatments, which may suggest risk for immune-related adverse events (irAEs). The following are not exclusionary: vitiligo, childhood asthma that has resolved, type 1 diabetes, residual hypothyroidism that required only hormone replacement, or psoriasis that does not require systemic treatment; (6) Has participated in a study of an investigational agent or an investigational device within 4 weeks of the randomization date or five half-lives (whichever is longer), though patients who have received or are enrolled in a study involving treatment with an investigational immunoPET reagent are not excluded; (7) Receipt of a live vaccine within 28 days of the randomization date; (8) Has had prior systemic anti-cancer immunotherapy for CSCC. Examples of immune modulating agents include but are not limited to blockers of CTLA-4, 4-1 BB (CD137), or OX-40, therapeutic vaccines, anti-PD-1/PD-L1 or PI3Kδ inhibitors; (9) Immunosuppressive corticosteroid doses (>10 mg prednisone daily or equivalent) within 4 weeks prior to the first dose of REGN2810/placebo (NOTE: Patients who require brief course of steroids (e.g., prophylaxis for imaging assessments due to hypersensitivity to contrast agents) are not excluded. People taking steroids for physiologic replacement (i.e., adrenal insufficiency) are NOT excluded); (10) Has received treatment with an approved anticancer systemic therapy within 4 weeks of the randomization date or has not yet recovered (i.e., ≤grade 1 or baseline) from any acute toxicities except for laboratory changes as described in inclusion criteria 6, 7, and 8 (NOTE: Patients receiving bisphosphonates or denosumab are not excluded); (11) Prior allogeneic stem cell transplantation, or autologous stem cell transplantation; (12) Patients who have permanently discontinued anti-cancer immune modulating therapies due to drug-related toxicity; (13) Encephalitis, meningitis, or uncontrolled seizures in the year prior to screening/enrollment; (14) Patients with myocardial infarction within 6 months prior to the randomization date; (15) Any infection requiring hospitalization and/or intravenous antibiotic therapy within 2 weeks of the randomization date; (16) Active tuberculosis; (17) Uncontrolled infection with human immunodeficiency virus (HIV), hepatitis B or hepatitis C virus (HBV or HCV) infection; or diagnosis of immunodeficiency. (NOTES: • Patients with known HIV infection who have controlled infection (undetectable viral load (HIV RNA PCR) and CD4 count above 350, either spontaneously or on a stable antiviral regimen) are permitted. For patients with controlled HIV infection, monitoring will be performed per local standards. • Patients will be tested for HBV and HCV at screening. • Patients with HBV (hepatitis B surface antigen positive; HepBsAg+) who have controlled infection (serum HBV DNA PCR that is below the limit of detection AND receiving anti-viral therapy for HBV) are permitted. Patients with controlled infections must undergo periodic monitoring of HBV DNA. Patients must remain on anti-viral therapy for at least 6 months beyond the last dose of investigational study drug. • Patients who are HCV antibody positive (HCV Ab+) who have controlled infection (undetectable HCV RNA by PCR, either spontaneously or in response to a successful prior course of anti-HCV therapy) are permitted; (18) History of immune related pneumonitis within the last 5 years; (19) History of interstitial lung disease (e.g., idiopathic pulmonary fibrosis, organizing pneumonia) or active, noninfectious pneumonitis that required immune-suppressive doses of glucocorticoids to assist with management. A history of radiation pneumonitis in the radiation field is permitted as long as pneumonitis resolved≥6 months prior to the randomization date; (20) History of documented allergic reactions or acute hypersensitivity reaction attributed to antibody treatments; (21) Known hypersensitivity or allergy to any of the excipients in the REGN2810 drug product; (22) Patients with a history of solid organ transplant (patients with prior corneal transplant(s) are not excluded); (23) Any medical co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study; (24) Known psychiatric or substance abuse disorders that would interfere with participation with the requirements of the study; (25) Member of the clinical site study team or his/her immediate family; (26) Women with a positive serum β-human chorionic gonadotropin (HCG) pregnancy test at the screening/baseline visit. If positive, pregnancy must be ruled out by ultrasound for patient to be eligible; (27) Breastfeeding women; (28) Women of childbearing potential (WOCBP) or sexually active men, who are unwilling to practice highly effective contraception prior to the first dose of study therapy, during the study, and for at least 180 days after the last dose. Highly effective contraceptive measures include: a. Stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; b. Intrauterine device (IUD); intrauterine hormone-releasing system (IUS); c. Bilateral tubal ligation; d. Vasectomized partner; and/or e. Sexual abstinence. WOCBP are defined as females who have had 1 episode of menses and have not yet reached menopause or have become surgically sterile, as below. Menopause is defined as at least 12 consecutive months without any episode of menses (not hormonally induced). Postmenopausal women must be amenorrheic for at least 12 months in order not to be considered of childbearing potential. Pregnancy testing and contraception are not required for women with documented hysterectomy, bilateral oophorectomy, or tubal ligation. Sexual abstinence is considered a highly effective method only if defined as refraining from heterosexual intercourse during the entire period of risk associated with the study treatments. Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

### Study Variables

The primary endpoint of this study is DFS, defined as time from randomization to the first documented disease recurrence (local, regional and/or distant) or death due to any cause. For patients who do not have a tumor recurrence or death, DFS will be censored on the date of last disease assessment. The estimated time frame for DFS assessment is up to approximately 54 months.

Secondary endpoints of this include: • overall survival, defined as time from randomization to the date of death. A patient who has not died will be censored on the last known date as alive. The estimated time frame for OS assessment is up to approximately 78 months; • Freedom from locoregional recurrence, defined as time from randomization to the date of first locoregional recurrence (LRR). Patients who died without a preceding LRR will be censored on the date of death. For patients who do not have a LRR or death, FFLRR will be censored on the date of last disease assessment. The estimated time frame for FFLRR assessment is up to approximately 54 months; • Freedom from distant recurrence, defined as time from randomization to the date of first distant recurrence (DR). Patients who died without a preceding DR will be censored on the date of death. For patients who do not have a DR or death, FFDR will be censored on the date of last disease assessment. The estimated time frame for FFDR assessment is up to approximately 54 months; • Cumulative occurrence of SPTs for each patient from randomization to occurrence of first primary endpoint event or end of study. The estimated time frame of assessment for cumulative occurrence of SPTs is up to approximately 54 months; • Safety, as measured by the incidence and severity of treatment-emergent adverse events (TEAE), deaths, and laboratory abnormalities. The estimated time frame for safety assessments is up to approximately 78 months.

Pharmacokinetic (PK) variables are REGN2810 concentrations at each time point. Samples in this study are collected using a sparse sampling schedule (e.g., only 1 blood sample for drug concentration measurement is collected at any single clinic visit).

### Study Design

This study is a randomized, double-blind, placebo-controlled phase 3 trial evaluating REGN2810 as adjuvant treatment for CSCC patients with features associated with high risk of recurrent disease, who have completed surgery and post-operative RT. The study population is targeted to patients who have completed surgery and post-operative RT for CSCC and have at least 1 factor that puts them at high risk for recurrence of CSCC. Patients are randomized 1:1 to 350 mg REGN2810 versus placebo every 3 weeks (Q3W) for up to 48 weeks. The primary endpoint is disease-free survival (DFS). For patients who experience documented disease recurrence on study, there is an option for subsequent REGN2810 therapy after the first recurrence. FIG. 1 provides a flow diagram with a general overview of this study.

REGN2810 is supplied as a liquid in sterile, single-use vials. Each vial contains REGN2810 at a concentration of 50 mg/mL. Placebo is prepared using the same formulation as that used for REGN2810 without the addition of active substance. Placebo is supplied as a liquid in sterile, single-use vials. REGN2810 350 mg or placebo is administered in an outpatient setting as a 30-minute (±10 minutes) IV infusion every 3 weeks.

The study includes two parts. Part 1 (Blinded) includes a screening period of up to 28 days prior to randomization, a treatment period of up to 48 weeks, and a follow-up period. During the treatment period (up to 48 weeks), patients undergo imaging assessments for tumor recurrence at the end of each 12-week cycle during the planned treatment period of approximately 1 year (48 weeks). Patients undergo post-treatment follow-up until disease recurrence or end-of study. Part 1 of the study supports the primary endpoint. Part 2 (Unblinded) includes optional REGN2810 treatment for patients on the placebo arm who experience disease recurrence, and optional subsequent treatment with REGN2810 for patients on the REGN2810 arm who experience disease recurrence≥3 months after completing 48 weeks of planned REGN2810 treatment.

Study Part 1 (Blinded): Patients may begin screening once they have completed surgery for CSCC and post-operative RT. Patients who fulfill the eligibility criteria and display high risk features on surgical pathology of their resected tumor sample are randomized 1:1 to receive REGN2810 350 mg or placebo, intravenously (IV). The first dose of REGN2810 or placebo will be administered within 5 days of randomization, and this will occur between 2 and 6 weeks after completion of RT. REGN2810 or placebo will be administered IV every 3 weeks (Q3W) for up to 48 weeks or until unacceptable toxicity, disease recurrence, death, or withdrawal of consent. Patients will be evaluated in clinic prior to each REGN2810 or placebo treatment. Cycle length is 12 weeks (4 treatments per cycle, on a Q3W schedule). The follow-up period begins after patients discontinue treatment, either due to the completion of the planned 48-week treatment period or premature discontinuation of the treatment for any other reason.

DFS is the primary endpoint of Part 1. For patients with high risk CSCC, patterns of failure include locoregional recurrence, distant recurrence, locoregional and distant recurrence, or death (Porceddu et al., J Clin Oncol, 36(13):1275-83, 2018). DFS was chosen as the primary endpoint of the study as it encompasses all of these patterns of failure.

To evaluate the efficacy of adjuvant REGN2810 for patients at high risk of disease recurrence, REGN2810 is compared to placebo in a 1:1 randomization. Because the current standard of care after RT is surveillance, placebo is the appropriate control arm to compare against REGN2810 to allow assessment of efficacy of REGN2810 as adjuvant treatment after surgery and RT.

Study Part 2 (Unblinded): Optional REGN2810 treatment for patients on the placebo arm who experience disease recurrence and optional subsequent treatment with REGN2810 for patients on the REGN2810 arm who experience disease recurrence≥3 months after completing 48 weeks of planned REGN2810 treatment. Patients may be treated for up to 96 weeks.

Patients assigned to the placebo group in Part 1 are eligible to receive subsequent REGN2810 therapy open-label in Part 2 of the study if they meet the following criteria for demonstrating disease recurrence: (i) have documentation of disease recurrence; (ii) provide a separate written consent for subsequent REGN2810 therapy; (iii) have not discontinued placebo treatment due to unacceptable toxicity (if a patient discontinues study treatment due to unacceptable toxicity and then, due to unblinding, is found to have been on placebo, the patient will not have the opportunity to receive REGN2810 in Part 2 because such a patient would have met criteria for permanent study treatment discontinuation); and (iv) repeat screening procedures continue to meet study eligibility criteria (with exception of select eligibility criteria).

Patients assigned to the REGN2810 group in Part 1 are also eligible to receive subsequent REGN2810 therapy open-label in Part 2 of the study if they meet the following criteria for demonstrating disease recurrence: (i) documentation of disease recurrence at least 3 months (90 days±3 days) after completion of 48 weeks of planned REGN2810 treatment (even if 1 or more doses of planned REGN2810 treatment were missed during the 48-week treatment period); (ii) provide a separate written consent for subsequent REGN2810 treatment in Part 2; (iii) prior REGN2810 was not discontinued due to unacceptable toxicity; and (iv) repeat screening procedures continue to meet study eligibility criteria (with exception of select eligibility criteria).

Patients eligible for subsequent REGN2810 therapy in Part 2 of the study may receive REGN2810 350 mg Q3W for up to 96 weeks (in Part 2) or until disease progression, unacceptable toxicity, withdrawal of consent, death, or lost to follow-up.

The severity of AEs (including test findings classified as AEs) will be graded using the NCI-CTCAE grading system (NCI-CTCAE v5). Adverse events not listed in the NCI-CTCAE v5 will be graded according to the scale set forth above in Table 1.

### Concomitant Medications and Procedures

Prohibited Medications and Procedures: While participating in this study, a patient may not receive any of the following from the time of informed consent to the end of the follow-up period, unless otherwise specified below: (a) Standard or investigational agent for treatment of a tumor other than REGN2810 or placebo; (b) Agents that block the PD-1/PD-L1 pathway (other than for patients who are assigned to receive REGN2810 in the study); (c) Radiation therapy; and (d) Live vaccines for at least 3 months after the last dose of study drug.

Permitted Medications and Procedures: The following medications and procedures are permitted, under the following conditions: (a) any medication required to treat an AE and/or irAE, including systemic corticosteroids, (b) systemic corticosteroids for physiologic replacement (even if >10 mg/day prednisone equivalents), (c) a brief course of corticosteroids for prophylaxis or for treatment of non-autoimmune conditions, (d) bisphosphonates and denosumab, (e) physiologic replacement doses of systemic corticosteroids, even if >10 mg/day prednisone equivalents, (f) oral contraceptives, hormone-replacement therapy, or other maintenance therapy may continue, (g) acetaminophen at doses≤2 g/day, (h) surgical resection of non-index lesions, if clinically indicated, and (i) other medications and procedures may be permitted on an individual basis. Because this is an adjuvant study, surgery is not planned. However, if surgery for any emergent medical issue(s) is clinically indicated for an individual patient, this is allowed.

### Procedures and Assessments

Screening/baseline procedures include: coagulation testing, serum β-HCG assay, brain imaging (computed tomography [CT] or magnetic resonance imaging [MRI]), and screening for hepatitis B virus (HBV), hepatitis C virus (HCV), and human immunodeficiency virus (HIV).

Efficacy procedures include: radiologic imaging assessments for tumor recurrence. Computed tomography imaging of the chest, abdomen, and pelvis are required at each imaging assessment. Imaging of the head and neck are obtained for patients with resected HN lesions. Biopsy of tumors is performed when feasible to obtain pathologic (histologic or cytologic) evidence of recurrent disease or SPT. For recurrent lesions, the pattern of failure is assessed, with recurrence defined as ≥1 lesion that can be categorized as local, regional, or distant recurrence.

Safety procedures include: comparison of safety and tolerability of REGN2810 with placebo evaluated via adverse event (AE) capturing, physical examination (complete or limited), weight, 12-lead electrocardiogram (ECG), vital sign assessments, and laboratory testing, including hematology, blood chemistry, and urinalysis.

PK/drug concentrations: PK samples are collected for assessment of REGN2810 concentrations in serum.

### Results

It is expected that administration of REGN2810 as adjuvant treatment for CSCC patients who have completed surgery and post-operative RT and are at high risk of recurrence leads to reduced risk of subsequent disease recurrence or zero incidence of subsequent disease recurrence. Adjuvant REGN2810 treatment administered to high risk CSCC patients after surgery and RT is also expected to improve disease control.

### Example 2: Clinical Trial of Anti-PD-1 Antibody Administered as a Neoadjuvant Treatment for Stage II to IV (M0) Cutaneous Squamous Cell Carcinoma (CSCC)

This study is a phase 2, single-arm, open label, multicenter study for patients with stage II to IV (M0) CSCC who are candidates for surgery, but who have an increased risk of recurrence and/or risk of disfigurement or loss of function.

The exemplary anti-PD-1 antibody used in this study is REGN2810 (also known as cemiplimab, or H4H7798N as disclosed in U.S. Pat. No. 9,987,500), which is a fully human monoclonal anti-PD-1 antibody comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising SEQ ID NOs: 3-6, AAS, and SEQ ID NO: 8.

### Study Objectives

A primary objective of the study is to evaluate the efficacy of neoadjuvant treatment with REGN2810 as measured by pathologic complete response (pCR) rate per independent central pathology review.

Secondary objectives of the study include: (1) to evaluate the efficacy of neoadjuvant REGN2810 on measures of disease response, including (a) major pathologic response (mPR) rate per independent central pathology review, (b), pCR rate and mPR rate per local pathology review, and (c) objective response rate (ORR) prior to surgery, according to local assessment using RECIST 1.1; (2) to evaluate the efficacy of neoadjuvant REGN2810 on event free survival (EFS), disease free survival (DFS), and overall survival (OS); (3) to evaluate the safety profile of neoadjuvant REGN2810; (4) to assess change in surgical plan (ablative and reconstructive procedures) from the screening period to definitive surgery, both according to investigator review and independent surgical expert review; and (5) to assess change in post-surgical management plan (radiation, chemoradiation, or observation) from the screening period to post-surgery pathology review, both according to investigator review and independent surgical expert review.

Exploratory objectives of the study include: (1) to explore baseline tumor markers for associations with treatment responses, peripheral and tumor measures associated with REGN2810 mechanism of action and discovery of other potential predictive markers of efficacy or safety; (2) describe patterns of failure (locoregional versus distant) in patients who experience disease recurrence; (3) to evaluate the cost implication due to changes in surgical plan during screening period versus actual surgical procedure performed: (4) to evaluate the cost implication due to changes in post-surgical management plan during screening period versus actual post-surgical management; (5) to assess the immunogenicity of REGN2810; and (6) assess health-related quality of life in patients with CSCC who receive neoadjuvant REGN2810.

### Study Duration

The duration of Part 1 of the study (treatment period before surgery) is up to 12 weeks. The duration of Part 2 of the study (optional post-surgery treatment period) is up to 48 weeks. The follow-up period is up to 3 years. Patients will be followed until disease recurrence or end of study, whichever occurs first.

### Study Population

Approximately 76 patients will be enrolled. The target population will consist of adult patients with stage III to IV (M0) CSCC of the head/neck, extremity, or trunk, and selected patients with stage II CSCC, for whom surgery would be recommended in routine clinical practice.

Inclusion Criteria: A patient must meet the following criteria to be eligible for inclusion in the study: (1) at least 18 years of age; (2) stage I to IV (M0) CSCC, for which surgery would be recommended in routine clinical practice. For stage II patients, lesion must be ≥3 cm at the longest diameter (NOTE: Staging is defined according to the AJCC 8th edition for HN tumors (Amin MB, American Joint Committee on C, American Cancer S. AJCC cancer staging manual. 8th ed. Springer International Publishing; 2017) and according to the UICC 9th edition for non-HN tumors (O'Sullivan B, Union for International Cancer C. UICC manual of clinical oncology. 9th ed. John Wiley & Sons, Ltd; 2015); (3) at least 1 lesion that is measurable by RECIST 1.1: (4) Eastern Cooperative Oncology Group (ECOG) performance status of 0 or 1; (5) adequate organ and bone marrow function documented by (a) hemoglobin>9.0 g/dL; (b) absolute neutrophil count (ANC)>1.5×10⁹/L; (c) platelet count>75×10⁹/L; (d) serum creatinine<1.5 upper limit of normal (ULN) or estimated creatinine clearance (CrCl)>30 mL/min; (e) adequate hepatic function (total bilirubin<1.5×upper limit of normal (ULN); aspartate aminotransferase (AST) and alanine aminotransferase (ALT) both <3×ULN; alkaline phosphatase (ALP)<2.5×ULN) (NOTE: For patients with Gilbert's syndrome, total bilirubin≤3×ULN. Gilbert's syndrome must be documented appropriately as past medical history.); (6) willing and able to comply with clinic visits and study-related procedures; (7) willing and able to provide informed consent signed by study patient or legally acceptable representative; and (8) able to understand and complete study-related questionnaires.

Exclusion Criteria: A patient who meets any of the following criteria will be excluded from the study: (1) Solid malignancy within 5 years of the projected enrollment date, or hematologic malignancy (including chronic lymphocytic leukemia [CLL]) at any time (NOTE: Patients with nonmelanoma skin cancer that has undergone potentially curative therapy, or in situ cervical carcinoma or in-situ prostate cancer with non-detectable prostate specific antigen or any other tumor that has been treated are not excluded if the patient is deemed to be in complete remission for at least 2 years prior to enrollment, and no additional therapy is required during the study period); (2) distant metastatic disease (M1), visceral and/or distant nodal; (3) prior radiation therapy for CSCC; (4) patients with a condition requiring corticosteroid therapy (>10 mg prednisone/day or equivalent) within 14 days of the first dose of study drug (NOTE: Physiologic replacement doses are allowed even if they are >10 mg of prednisone/day or equivalent, as long as they are not being administered for immunosuppressive intent. Inhaled or topical steroids are permitted, provided that they are not for treatment of an autoimmune disorder.); (5) patients with active, known, or suspected autoimmune disease that has required systemic therapy within 5 years of the projected enrollment date (NOTE: Patients with vitiligo, type I diabetes mellitus, and endocrinopathies (including hypothyroidism due to autoimmune thyroiditis) only requiring hormone replacement, childhood asthma that has resolved, or psoriasis that does not require systemic treatment are permitted.); (6) History of interstitial lung disease (e.g., idiopathic pulmonary fibrosis, organizing pneumonia) or active, noninfectious pneumonitis that required immune-suppressive doses of glucocorticoids to assist with management; (7) uncontrolled infection with human immunodeficiency virus (HIV), hepatitis B or hepatitis C virus (HBV or HCV) infection; or diagnosis of immunodeficiency (NOTES: (a) patients will be tested for HIV, HBV, and HCV at screening; (b) patients with known HIV infection who have controlled infection (undetectable viral load [HIV RNA measured via polymerase chain reaction] and CD4 count above 350 either spontaneously or on a stable antiviral regimen) are permitted. For patients with controlled HIV infection, monitoring will be performed per local standards; (c) patients with hepatitis B (HBsAg+) who have controlled infection (serum hepatitis B virus DNA measured via polymerase chain reaction that is below the limit of detection AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug; (d) patients who are hepatitis C virus antibody positive (HCV Ab+) who have controlled infection (undetectable HCV RNA by polymerase chain reaction either spontaneously or in response to a successful prior course of anti-HCV therapy) are permitted; (8) active tuberculosis; (9) myocardial infarction within 6 months of enrollment; (10) any medical co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study; (11) documented allergic or acute hypersensitivity reaction attributed to antibody treatments; (12) prior treatment with anti-cancer systemic therapy within the last 3 years prior to projected enrollment date; (13) ay prior treatment with an anti-PD1/PD-L1 agent; (14) has participated in a study of an investigational agent or an investigational device within 4 weeks of enrollment; (15) women with a positive serum chorionic gonadotropin HCG pregnancy test at the screening/baseline visit. Breastfeeding women are also excluded; (16) women of childbearing potential (as defined below) and sexually active men who are unwilling to practice highly effective contraception prior to the first dose of study therapy, during the study, and for at least 6 months after the last dose. Highly effective contraceptive measures include: (a) stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; (b) intrauterine device (IUD); intrauterine hormone-releasing system (IUS); (c) bilateral tubal ligation; (d) vasectomized partner (provided that the male vasectomized partner is the sole sexual partner of the women of childbearing potential (WOCBP) study participant and that the vasectomized partner has obtained medical assessment of surgical success for the procedure); and/or (e) sexual abstinence (as defined below); (17) receipt of a live vaccine within 28 days of enrollment; (18) prior allogeneic stem cell transplantation, or autologous stem cell transplantation; (19) recipient of a solid organ transplant (other than corneal transplants); (20) diagnosis of squamous cell carcinoma of unknown (or occult) primary; (21) patients who are committed to an institution by virtue of an order issued either by the judicial or the administrative authorities; and (22) member of the clinical site study team or his/her immediate family, unless prior approval granted by the sponsor.

Women of childbearing potential are defined as women who are fertile following menarche until becoming postmenopausal (as defined below), unless permanently sterile. Permanent sterilization methods include hysterectomy, bilateral salpingectomy, and bilateral oophorectomy. A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to Clinical Trial Facilitation Group (CTFG) guidance. Pregnancy testing and contraception are not required for women with documented hysterectomy or tubal ligation.

Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient. Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

### Study Variables

For endpoints measuring pCR, mPR, and ORR (Part 1 of the study), patients will be assessed at the time of surgery (12 weeks). Event free survival (EFS) and OS will be assessed from the first dose of neoadjuvant REGN2810 until completion of follow-up. Disease free survival (DFS) will be assessed from surgery until completion of follow-up. Event free survival (EFS), DFS, and OS assessment will continue until all enrolled patients have completed follow-up, a total duration of approximately 4 years and 3 months.

Primary endpoints for this study include: pCR rate assessed by independent central pathology review.

Secondary endpoints for this study include: (1) mPR rate assessed by independent central pathology review; (2) pCR rate and mPR rate assessed by local pathology review; (3) ORR prior to surgery, according to investigator assessment using RECIST 1.1; (4) event free survival (EFS); (5) disease free survival (DFS); (6) overall survival (OS); (7) safety and tolerability as measured by the incidence of adverse events (AEs), serious adverse events (SAEs), deaths, and laboratory abnormalities; (8) change in surgical plan in the screening period versus actual surgery after neoadjuvant REGN2810; and (9) change in post-surgical management plan in the screening period versus actual post-surgical management.

Exploratory variables for this study include: (1) patterns of failure in patients with local, regional, or distant disease recurrence as measured by descriptive statistics; (2) change in estimated costs due to change in surgical plan during screening period versus actual surgical procedure performed after neoadjuvant REGN2810 (3) change in estimated costs due to the change in post-surgical management plan during screening period versus actual post-surgical management; (4) incidence of ADA for REGN2810; and (5) health-related quality of life, as assessed using the EORTC QLQ-C30.

Pharmacokinetic (PK) variables include REGN2810 concentrations concentration in serum over time.

Immunogenicity variables include anti-drug antibody (ADA) status, dose titer, and time-point/visit.

Safety variables include (1) vital signs; (2) physical examination results; (3) electrocardiogram (ECG) results; (4) clinical laboratory results; (5) adverse events (AEs); and (6) immune-related adverse events (irAEs).

Pathologic complete response (pCR) is defined as absence of viable cancer cells in the surgical pathology sample.

Major pathologic response (mPR) is defined as ≤10% viable cancer cells in the surgical pathology sample, in patients who have not achieved pCR.

Objective response rate (ORR) will be assessed by the investigator using RECIST 1.1 (Eur J Cancer 2009; 45(2):228-47).

Event free survival (EFS) is defined as time from first dose of neoadjuvant REGN2810 to any of the following events: progression of disease that precludes surgery, inability to undergo complete resection (R0 or R1), disease recurrence (local, regional, or distant) for patients who undergo complete resection (R0 or R1), or death due to any cause.

Disease free survival (DFS) is defined as time from surgery to first recurrence (local, regional, or distant) or death due to any cause, for patients who are free of disease (R0 or R1 resection) at completion of surgery.

Overall survival (OS) will be measured as time from first dose of neoadjuvant REGN2810 to death due to any cause.

### Study Design

This study is a single-arm, open label, multicenter phase 2 study for patients with stage I to IV (M0) CSCC who are candidates for surgery, but who have an increased risk of recurrence and/or risk of disfigurement or loss of function.

The study consists of 2 parts:

Part 1 (neoadjuvant): A screening period of up to 28 days, a treatment period of up to 12 weeks, and surgery after up to 12 weeks of treatment. Part 1 of the study supports the primary endpoint.

Part 2 (adjuvant): Optional post-surgery REGN2810 treatment for up to 48 weeks (or radiation therapy, or observation only, at investigator discretion).

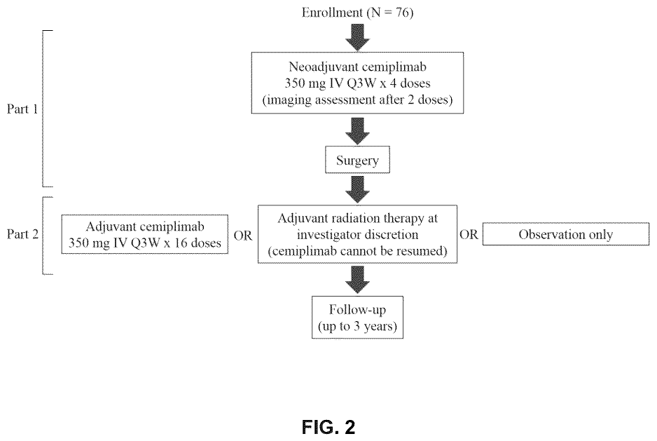

After Part 2 of the study, patients will be followed for a period of up to 3 years. FIG. 2 provides a flow diagram with a general overview of this study.

REGN2810 supplied as a sterile liquid in single-use glass vials. Each vial contains REGN2810 at a concentration of 50 mg/mL. REGN2810 350 mg is administered as an IV infusion over 30 minutes (±10 minutes) every 3 weeks (Q3W) for up to 12 weeks (up to 4 doses) prior to surgery in Part 1 and (optional) up to 48 weeks (up to 16 doses) after surgery in Part 2.

Study Part 1: Patients with stage I to IV (M0) CSCC with planned surgery and who fulfill the eligibility criteria will receive REGN2810 350 mg intravenously (IV) every 3 weeks (Q3W) for up to 12 weeks (up to 4 doses), or until unacceptable toxicity, disease progression, or withdrawal of consent. Patients will be evaluated in clinic prior to each dose and will undergo tumor response imaging assessment prior to receiving the third dose of REGN2810 (day 43±3) and prior to surgery (day 85). The window for surgery is from day 75 through day 100. If a patient meets criteria to discontinue REGN2810 during the 12-week neoadjuvant period, the treating physician may divert the patient to surgery at an earlier time. Following surgical tumor resection, the primary endpoint (pCR rate) will be assessed by an independent central pathology review committee.

Study Part 2: Patients will have the option to receive adjuvant REGN2810 treatment (350 mg IV Q3W) following surgery for up to 48 weeks (up to 16 doses) or until unacceptable toxicity, disease recurrence, or withdrawal of consent. The first dose of adjuvant treatment will occur 3 weeks (±3 days) after the end of treatment in Part 1 (EOT1). At the investigator's discretion, patients may alternatively receive adjuvant radiation therapy (concurrent or subsequent REGN2810 treatment not allowed) or enter an observation-only period. During Part 2 of the study, patients will be evaluated in clinic every 15 weeks. Patients receiving adjuvant REGN2810 will undergo complete assessments as described in the schedule of events, while patients who do not receive adjuvant REGN2810 will only undergo imaging assessments following a parallel schedule.

Follow-up: Follow-up will begin after a patient has completed Part 1 and Part 2 of the study without disease progression (pre-surgery) or disease recurrence (post-surgery). Patients will be evaluated in clinic for up to 3 additional years.

### Concomitant Medications and Procedures

Prohibited Medications and Procedures: While participating in this study (not including survival follow-up), a patient may not receive any of the following medications from the time of informed consent to the end of the follow-up period, unless otherwise specified below: (1) standard or investigational agent (other than REGN2810) for treatment of a tumor, with the exception of [ . . . ]; and (2) live vaccines for at least 3 months after the last dose of study drug.

Permitted Medications and Procedures: The following medications and procedures will be permitted, under the following conditions: (1) any medication required to treat an AE and/or irAE, including systemic corticosteroids; (2) systemic corticosteroids for physiologic replacement (even if >10 mg/day prednisone equivalents); (3) a brief course of corticosteroids for prophylaxis or for treatment of non-autoimmune conditions; (4) oral contraceptives, hormone-replacement therapy, or other maintenance therapy may continue; (5) acetaminophen at doses≤2 g/day; (5) surgical resection of pre-malignant lesions or basal cell carcinoma (BCC) lesions; and (6) other medications and procedures may be permitted on an individual basis by the investigator and in consultation with the sponsor. Radiation therapy (with concurrent anti-cancer cytotoxic chemotherapy therapy and/or epidermal growth factor receptor-directed therapy) is permitted in the adjuvant portion of the study, at the discretion of the investigator. Such patients will be followed for disease recurrence but will not receive further REGN2810 on study.

### Procedures and Assessments

Screening/baseline procedures include: screening for hepatitis B virus (HBV), hepatitis C virus (HCV), and human immunodeficiency virus (HIV), height measurement, serum β-human chorionic gonadotropin (HCG) testing (for women of childbearing potential).

Efficacy procedures include: (1) Evaluation of surgically resected tumors for pathologic response; (2) radiologic imaging assessments for tumor response (neoadjuvant portion of the study) and for disease recurrence (adjuvant portion of the study and follow-up); (3) imaging of externally visible lesions, supplemented with digital medical photography; (4) biopsy of tumors will to obtain histologic or cytologic evidence of disease recurrence or evidence of a second primary tumor (SPT); and (5) assessment of the pattern of failure for recurrent lesions, with recurrence defined as ≥1 lesion that can be categorized as local, regional, or distant recurrence.

Safety procedures include: capturing of adverse event (AE), physical examination (complete or limited), assessment of weight, recording of a 12-lead electrocardiogram (ECG), vital sign assessments, and laboratory testing, including hematology, blood chemistry, and urinalysis.

Laboratory testing procedures include: (1) blood chemistry: sodium, potassium, chloride, carbon dioxide (bicarbonate), calcium, glucose (fasting or non-fasting), albumin, total protein (serum), creatinine, blood urea nitrogen (BUN), aspartate aminotransferase (AST), alanine aminotransferase (ALT), alkaline phosphatase, total bilirubin); hematology: hemoglobin, white blood cells, platelet count, neutrophils, lymphocytes); (3) urinalysis: pH, specific gravity, ketones, glucose, blood, spot urine protein; and (4) other laboratory tests: (a) HBV, HCV, HIV testing; (b) pregnancy test: Serum β-HCG or urine β-HCG; (c) thyroid-stimulating hormone (TSH) with reflex T3 and free T4; and (d) coagulation, using measurements of INR and aPTT.

### Results

For Phase 1 of the study, it is expected that administration of REGN2810 as neoadjuvant treatment prior to surgery will result in a significant pathologic complete response (pCR) rate in patients with CSCC.

For Phase 2 of the study, it is expected that administration of REGN2810 as adjuvant treatment for CSCC patients who have completed surgery and post-operative RT and are at high risk of recurrence leads to reduced risk of subsequent disease recurrence or zero incidence of subsequent disease recurrence. Adjuvant REGN2810 treatment administered to high risk CSCC patients after surgery and RT is also expected to improve disease control.

### Example 3: Clinical Trial of Anti-PD-1 Antibody Administered in Patients with Metastatic or Unresectable, Advanced Cutaneous Squamous Cell Carcinoma (CSCC)

This study is a phase 2, non-randomized, 5-group pivotal trial for patients with advanced CSCC.

The exemplary anti-PD-1 antibody used in this study is REGN2810 (also known as cemiplimab, or H4H7798N as disclosed in U.S. Pat. No. 9,987,500), which is a fully human monoclonal anti-PD-1 antibody comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising SEQ ID NOs: 3-6, AAS, and SEQ ID NO: 8.

### Study Groups

Group 1 consists of patients with metastatic (nodal or distant) CSCC treated with REGN2810 (3 mg/kg/dose, IV, once every two weeks).

Group 2 consists of patients with unresectable locally advanced CSCC, treated with REGN2810 (3 mg/kg/dose, IV, once every two weeks).

Group 3 consists of patients with metastatic (nodal or distant) CSCC treated with REGN2810 (350 mg/dose, IV, once every three weeks).

Group 4 consists of patients with advanced CSCC [metastatic (nodal or distal) or unresectable locally advanced] treated with REGN2810 (600 mg/dose, IV, once every four weeks).

Group 5 consists of patients with advanced CSCC (metastatic or locally advanced) treated with REGN2810 (438 mg/dose, SC) and three weeks later with REGN2810 (350 mg/dose, IV, once every three weeks).

An overview of the disease stages for the five study groups is provided in Table 3.

TABLE 3
Disease Stages for Study Groups 1-5

| Group | Disease Stage |
|---|---|
| 1 | metastatic CSCC (includes patients with both nodal |
| | metastatic and distant metastatic disease) |
| 2 | unresectable locally advanced CSCC (inoperable, |
| | medical contraindication to surgery or radiation, |
| | or no disease control with these treatments) |
| 3 | metastatic CSCC (includes patients with both nodal |
| | metastatic and distant metastatic disease) |
| 4 | advanced CSCC (metastatic [nodal or distant] or |
| | locally advanced) |
| 5 | advanced CSCC (either metastatic CSCC or locally |
| | advanced) |

### Study Objectives

Primary objectives of this study for Groups 1 to 4 include: to estimate the clinical benefit (overall response rate by central review) of REGN2810 monotherapy for patients with metastatic (nodal or distant) CSCC, or unresectable CSCC. Objectives of this study for Group 5 include: (1) to measure concentrations of REGN2810 in serum after subcutaneous (SC) administration to assess the subcutaneous bioavailability of REGN2810 and (2) to evaluate the safety, tolerability, and PK of a single dose of subcutaneous REGN2810.

Secondary objectives of the study include: (1) to estimate the objective response rate (ORR) according to investigator review; (2) estimate the duration of response, progression-free survival (PFS), and overall survival (OS) by central and investigator review; (3) to estimate the complete response (CR) rate by central review; (4) to assess the safety and tolerability of REGN2810; (5) to assess the pharmacokinetics (PK) of REGN2810; (6) to assess the immunogenicity of REGN2810; and (7) to assess the impact of REGN2810 on quality of life using European Organisation for Research and Treatment of Cancer Quality of Life Questionnaire Core 30 (EORTC QLQ-C30, see Young et al. European Journal of Cancer, 1999, 35(13):1773-82).

Secondary objectives of the study for Group 4 further include: to assess ORR according to ¹⁸F-fluorodeoxyglucose positron emission tomography (¹⁸F-FDG-PET) using EORTC criteria.

Further study objectives for Groups 2 and 4 include: (1) to assess the pharmacodynamic effects of REGN2810 in tumor biopsies obtained at baseline, during treatment, and at progression in CSCC patients treated with REGN2810; (2) number and distribution of tumor-infiltrating lymphocytes (TILs) (CD8⁺ T cells, CD4⁺ T cells, T regulatory cells, and tissue permitting, other subtypes such as B cells, myeloid-derived cells, NK cells, etc.); (3) expression levels (mRNA and/or protein) of programmed death ligand 1 (PD-L1), glucocorticoid-induced TNFR family related gene (GITR), and lymphocyte activation gene-3 (LAG-3), and possibly other check point modulator; (4) mutations in known oncogenes and potential tumor neoantigens; and (5) tumor mutation burden.

### Study Duration

The duration of the study includes a screening period (up to 4 weeks) for all five groups. Groups 1 and 2 each receive up to 96 weeks of treatment. Group 3 receives up to 54 weeks of treatment. Group 4 receives up to 48 weeks of treatment. Group 5 receives up to 54 weeks of treatment. All groups are eligible for up to 2 years of retreatment.

All groups will receive up to approximately 1.5 years of follow-up.

### Study Population

The study includes eligible patients with metastatic (nodal and/or distant) CSCC (Groups 1 and 3) and unresectable locally advanced CSCC (Group 2). Group 3 for metastatic CSCC opens only after enrollment to Group 1 is complete. Groups 4 and 5 enroll patients with advanced CSCC, a term that encompasses both metastatic (nodal or distant) CSCC and locally advanced CSCC.

Inclusion Criteria: A patient must meet the following criteria to be eligible for inclusion in the study: (1) histologically confirmed diagnosis of invasive CSCC; (a) concerning the tumor primary site: Patients for whom the primary site of squamous cell carcinoma was the dry red lip (vermillion) are not eligible. Patients with tumors arising on the cutaneous hairbearing (non-glabrous) lip with extension onto dry red lip (vermillion) may be eligible after communication with and approval from medical monitor. Patients for whom the primary site of squamous cell carcinoma was the anogenital area (penis, scrotum, and perianal region) are not eligible. Patients for whom the primary site is nose are only eligible if the investigator is able to establish unambiguously that the primary site was skin, not nasal mucosa with outward extension to skin; and (b) concerning tumor histology: Patients with mixed histologies (e.g., sarcomatoid, adenosquamous) generally will not be eligible. Patients with mixed histology in which the predominant histology is invasive CSCC (with only a minimal component of mixed histology) may be eligible, after communication with and approval from medical monitor; (2) at least one lesion that is measurable by study criteria. If a previously radiated lesion is to be followed as a target lesion, progression must be confirmed by biopsy after radiation therapy. Previously radiated lesions may be followed as non-target lesions if there is at least one other measurable target lesion; (a) for patients with metastatic (nodal or distant) CSCC (all Groups 1 and 3 patients and patients in Groups 4 and 5 with metastatic CSCC): There must be at least one baseline measurable lesion 10 mm in maximal diameter (1.5 cm for lymph nodes) according to Response Evaluation Criteria in Solid Tumors (RECIST) 1.1 criteria. In the case of patients with metastatic disease that does not meet target lesion criteria by RECIST 1.1 (e.g., bone only lesions, perineural disease; see Eisenhauer et al. Eur J Cancer 2009; 45: 228-247). With externally visible CSCC target lesion(s), bi-dimensional measurements by digital medical photography may be used (at baseline, perpendicular diameters must both be 10 mm). The patient would then be enrolled with the plan to measure externally visible target lesion(s) by photography with bi-dimensional measurements; the metastatic lesions that are not measurable by RECIST 1.1 criteria would be followed as non-target lesions on scans; (b) for patients with unresectable locally advanced CSCC (all Group 2 patients and patients in Groups 4 and 5 with locally advanced CSCC): There must be at least one measurable baseline lesion in which the longest diameter (LD) and the perpendicular diameter are both 10 mm if followed by digital medical photography. Non-measurable disease for Group 2 is defined as either unidimensionally measurable lesions, tumors with margins that are not clearly defined, or lesions with maximum perpendicular diameters less than 10 mm. Patients without measurable disease at baseline are not eligible for the study; (3) Eastern Cooperative Oncology Group (ECOG) performance status≤1 (ECOG PS 1 definition: Restricted in physically strenuous activity but ambulatory and able to carry out work of a light or sedentary nature, e.g., light house work, office work). Patients with ECOG PS>1 are ineligible; (4) 18 years old; (5) hepatic function; (a) total bilirubin 51.5×upper limit of normal (ULN; if liver metastases≤3×ULN). Patients with Gilbert's Disease and total bilirubin up to 3×ULN may be eligible after communication with and approval from the medical monitor; (b) transaminases≤3×ULN (or 55.0×ULN, if liver metastases); and (c) alkaline phosphatase (ALP)≤2.5×ULN (or ≤5.0×ULN, if liver or bone metastases). For patients with hepatic metastases who wish to enroll in Group 1, Group 3, Group 4, or Group 5: If transaminase levels (AST and/or ALT) are >3×but ≤5×ULN, total bilirubin must be 51.5×ULN. If total bilirubin is >1.5× but ≤3×ULN, both transaminases (AST and ALT) must be ≤3×ULN; (6) renal function: Serum creatinine≤1.5×ULN or estimated creatinine clearance (CrCl)>30 mL/min; (7) bone marrow function: (a) hemoglobin≥9.0 g/dL; (b) absolute neutrophil count (ANC)≥1.5×10⁹/L; and (c) platelet count≥75×10⁹/L; (8) ability to provide signed informed consent; (9) ability and willingness to comply with scheduled visits, treatment plans, laboratory tests, and other study-related procedures; (10) anticipated life expectancy>12 weeks; (11) patients with locally advanced CSCC: All Group 2 patients (and Groups 4 and 5 patients with locally advanced CSCC): Surgery must be deemed contraindicated in the opinion of a Mohs dermatologic surgeon, a head and neck surgeon, or plastic surgeon. A copy of the surgeon's consultation note from a clinical visit within 60 days of enrollment must be submitted. Acceptable contraindications in the surgeon's note include: (a) CSCC that has recurred in the same location after 2 or more surgical procedures and curative resection is deemed unlikely; (b) CSCCs with significant local invasion that precludes complete resection; (c) CSCCs in anatomically challenging locations for which surgery may result in severe disfigurement or dysfunction (e.g., removal of all or part of a facial structure, such as nose, ear, or eye; or requirement for limb amputation); and (d) other conditions deemed to be contraindicating for surgery must be discussed with the medical monitor before enrolling the patient; (12) patients with locally advanced CSCC (all Group 2 patients and Group 4 and 5 patients with locally advanced CSCC): Patients must be deemed as not appropriate for radiation therapy. Specifically, patients must meet at least one of the following criteria: (a) a patient previously received radiation therapy for CSCC, such that further radiation therapy would exceed the threshold of acceptable cumulative dose, per the radiation oncologist. A copy of the radiation oncologist's consultation note, from a clinical visit within 60 days of enrollment, must be submitted; (b) judgment of radiation oncologist that such tumor is unlikely to respond to therapy. A copy of the radiation oncologist's consultation note, from a clinical visit within 60 days of enrollment, must be submitted; and (c) a clinic note from the investigator indicating that an individualized benefit:risk assessment was performed by a multidisciplinary team (consisting of, at minimum, a radiation oncologist AND EITHER a medical oncologist with expertise in cutaneous malignancies OR a dermato-oncologist, OR a head and neck surgeon) within 60 days prior to enrollment in the proposed study, and the radiation therapy was deemed to be contraindicated. Acceptable contraindications to radiation therapy in the investigator's note for patients who have not received any prior radiation include: (i) CSCCs in anatomically challenging locations for which radiation therapy would be associated with unacceptable toxicity risk in the context of the patient's overall medical condition in the opinion of the multidisciplinary team (e.g., a neck tumor for which radiation therapy would result in potential need for a percutaneous gastrostomy tube). A copy of the investigator's consultation note documenting the multidisciplinary assessment must be submitted; and (ii) other conditions deemed to be contraindicating for radiation therapy must be discussed with the medical monitor before enrolling the patient; (13) all patients in either group must consent to provide archived or newly obtained tumor material (either formalin-fixed, paraffin-embedded [FFPE] block or 10 unstained or stained slides) for central pathology review for confirmation of diagnosis of CSCC. This material must be confirmed as received by the central lab prior to enrollment; (14) Group 2 (locally advanced CSCC patients) and Group 4 (locally advanced CSCC patients and metastatic CSCC patients) only: Patients must consent to undergo biopsies of CSCC lesions at baseline, cycle 1 day 29 (±3 business days), at time of tumor progression, and at other time points that may be clinically indicated in the opinion of the investigator; (15) all Group 2 patients, and only those Groups 4 and 5 patients with locally advanced CSCC: An investigator note which states that the natural history of the patient's advanced CSCC would likely be life-threatening within 3 years with currently available management options outside of a clinical trial.

Exclusion criteria: A patient who meets any of the following criteria will be excluded from the study: (1) ongoing or recent (within 5 years) evidence of significant autoimmune disease that required treatment with systemic immunosuppressive treatments, which may suggest risk for immune-related adverse events (irAEs). The following are not exclusionary: vitiligo, childhood asthma that has resolved, type 1 diabetes, residual hypothyroidism that required only hormone replacement, or psoriasis that does not require systemic treatment; (2) prior treatment with an agent that blocks the PD-1/PD-L1 pathway; (3) prior treatment with other immune modulating agents that was (a) within fewer than 4 weeks (28 days) prior to the first dose of REGN2810, or (b) associated with immune mediated adverse events that were grade 1 within 90 days prior to the first dose of REGN2810, or (c) associated with toxicity that resulted in discontinuation of the immune-modulating agent. Examples of immune modulating agents include therapeutic anti-cancer vaccines, cytokine treatments (other than G-CSF or erythropoietin), or agents that target cytotoxic T-lymphocyte antigen 4 (CTLA-4), 4-1 BB (CD137), PI 3-K-delta, or OX-40; (4) untreated brain metastasis(es) that may be considered active. (Note: patients with brain involvement of CSCC due to direct extension of invading tumor, rather than metastasis, may be allowed to enroll if they do not require greater than 10 mg prednisone daily, after discussion and approval of the medical monitor). Patients with previously treated brain metastases may participate provided that the lesion(s) is (are) stable (without evidence of progression for at least 6 weeks on imaging obtained in the screening period), and there is no evidence of new or enlarging brain metastases, and the patient does not require any immunosuppressive doses of systemic corticosteroids for management of brain metastasis(es) within 4 weeks of first dose of REGN2810; (5) immunosuppressive corticosteroid doses (>10 mg prednisone daily or equivalent) within 4 weeks prior to the first dose of REGN2810. Note: Patients who require brief course of steroids (e.g., as prophylaxis for imaging studies due to hypersensitivity to contrast agents) are not excluded; (6) active infection requiring therapy, including infection with human immunodeficiency virus, or active infection with hepatitis B virus (HBV) or hepatitis C virus (HCV); (7) history of non-infectious pneumonitis within the last 5 years. If pneumonitis was purely infectious in etiology, enrolling on protocol may be allowed after discussion with medical monitor; (8) grade≥3 hypercalcemia at time of enrollment; (9) any systemic anticancer treatment (chemotherapy, targeted systemic therapy, photodynamic therapy), investigational or standard of care, within 30 days of the initial administration of REGN2810 or planned to occur during the study period (Patients receiving bisphosphonates or denosumab are not excluded), radiation therapy within 14 days of initial administration of REGN2810 or planned to occur during the study period. Note: For patients with multiple CSCCs at baseline that are not designated by the investigator as target lesions, treatment of these non-target CSCCs with surgery may be permitted but must be discussed with the medical monitor prior to any surgical procedure; (10) history of documented allergic reactions or acute hypersensitivity reaction attributed to antibody treatments; (11) patients with allergy or hypersensitivity to REGN2810 or to any of the excipients must be excluded. Specifically, because of the presence of trace components in REGN2810, patients with allergy or hypersensitivity to doxycycline or tetracycline are excluded; (12) breast feeding; (13) positive serum pregnancy test (a false positive pregnancy test, if demonstrated by serial measurements and negative ultrasound, will not be exclusionary, upon communication with and approval from the medical monitor); (14) concurrent malignancy other than CSCC and/or history of malignancy other than CSCC within 3 years of date of first planned dose of REGN2810, except for tumors with negligible risk of metastasis or death, such as adequately treated BCC of the skin, carcinoma in situ of the cervix, or ductal carcinoma in situ of the breast, or low-risk early stage prostate adenocarcinoma (T1-T2aN0M0 and Gleason score 56 and PSA≤10 ng/mL) for which the management plan is active surveillance, or prostate adenocarcinoma with biochemical-only recurrence with documented PSA doubling time of >12 months for which the management plan is active surveillance (D'Amico 2005, Pham 2016). Patients with hematologic malignancies (e.g., chronic lymphocytic leukemia, CLL) are excluded; (15) any acute or chronic psychiatric problems that, in the opinion of the investigator, make the patient ineligible for participation; (16) continued sexual activity in men or women of childbearing potential who are unwilling to practice highly effective contraception prior to the initial dose, during the study, and for at least 6 months after the last dose of study drug. Highly effective contraceptive measures include: stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device; intrauterine hormone releasing system; bilateral tubal ligation; vasectomized partner, and sexual abstinence. Contraception is not required for men with documented vasectomy. Postmenopausal women must be amenorrheic for at least 12 months in order not to be considered of childbearing potential. Pregnancy testing and contraception are not required for women with documented hysterectomy or tubal ligation; (17) patients with a history of solid organ transplant (patients with prior corneal transplant(s) may be allowed to enroll after discussion with and approval from the medical monitor); (18) prior treatment with a BRAF inhibitor; (19) any medical co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study; (20) inability to undergo any contrast-enhanced radiologic response assessment. Notes regarding imaging options: A patient who is unable to undergo CT with iodinated contrast (e.g., due to contrast allergy) would not be excluded if his/her disease can be measured by MRI with gadolinium. A patient who is unable to undergo MRI with gadolinium would not be excluded if his/her disease can be measured by CT scan with contrast. Note regarding Groups 2, 4, and 5 (locally advanced CSCC patients only): In selected cases, a locally advanced CSCC patient (in Group 2, 4, or 5) who is unable to undergo any contrast enhanced radiographic imaging (neither CT with iodinated contrast nor MRI with gadolinium) may be eligible if the patient's disease can be comprehensively assessed with digital medical photography, after communication with and approval from medical monitor.

### Study Variables

Primary efficacy endpoints for this study include: ORR according to central review during the 12 treatment cycles (Groups 1 and 2) or 6 treatment cycles (Groups 3 and 4). Overall response rate will be assessed separately for patients with metastatic CSCC or unresectable locally advanced CSCC.

For patients with metastatic disease (Groups 1, 3, and 4) RECIST version 1.1 will be used to determine ORR (Eisenhauer, E A, et al. Eur J Cancer 2009; 45: 228-247). Patients in which all response assessments are performed on radiologic scans according to RECIST 1.1, the determination of the independent radiologic response assessment committee will serve as the central response assessment. Clinical or composite response criteria may be used for patients with externally visible target lesions, if all metastatic lesions are not measurable by RECIST (such as may occur in patients with bone-only metastases).

For patients with unresectable locally advanced disease (Groups 2 and 4) clinical response criteria will be used to determine ORR, for externally visible tumor(s) require bi-dimensional measurements according to World Health Organization (WHO) criteria. Composite response criteria will be used for patients who have both target lesions measurable by clinical response criteria and by RECIST 1.1 to determine ORR. In patients achieving a CR, tumor biopsies will be used in the final determination of complete versus PR.

Patients who are deemed not evaluable (NE) by RECIST version 1.1 (Groups 1, 3, and 4) or inevaluable by the clinical or composite response criteria (Group 2) will be considered as not reaching partial response (PR)/complete response (CR) for ORR.

Secondary endpoints for this study include: (1) ORR for Groups 1 through 5 by investigator assessments: (a) for Groups 1, 3, 4 (if metastatic), and Group 5 (if metastatic) patients in which all response assessments are performed on radiologic scans according to RECIST 1.1, the term “composite response assessment” is not applicable. The investigator's response assessment for such patients will be RECIST 1.1 assessment; (b) for Groups 2, 4 (if unresectable locally advanced), and Group 5 (if unresectable locally advanced) patients in which all response assessments are performed on photographs according to Clinical Response Criteria for Externally Visible Tumors, the term “composite response assessment” is not applicable. The investigator's response assessment for such patients will be according to Clinical Response Criteria for Externally Visible Tumors; (c) For patients in which target lesion response assessments are performed with both scans (according to RECIST 1.1) and photographs (according to Clinical Response Criteria for Externally Visible Tumors), the investigator's response assessment will be according to Composite Response Criteria; (d) Patients in Group 4 will receive PET-CT scans at baseline and at 6-month intervals. Investigator response assessments will be according to EORTC criteria Young et al. European Journal of Cancer, 1999., Vol. 35, No. 13, 1773-82; (2) duration of response; (3) PFS; (4) OS; (5) CR rate; (6) change in scores of patient-reported outcomes on EORTC QLQ-C30; (7) AEs; (8) REGN2810 concentrations in serum; and (9) anti-REGN2810 antibodies.

Exploratory outcome measures for this study include: (1) fold-change in mRNA expression of genes expressed in tumor tissue; (2) percent change in number of TILs (CD8⁺ T cells, CD4⁺ T cells, T regulatory cells, and tissue permitting, other subtypes such as B cells, myeloid-derived cells, NK cells, etc.) and descriptive change in distribution of TILs in respect to tumor tissue and stroma; (3) percent change in expression levels (mRNA and/or protein) of PD-L1, GITR, and LAG-3, and possibly other check-point modulators; (4) change in number and type of genetic mutations in known oncogenes and potential tumor neoantigens; and (5) change in tumor mutation burden.

Serum concentration of REGN2810 will be assessed at multiple time points throughout the study treatment and follow-up periods, and the PK will be analyzed. Pharmacokinetic variables may include, but are not limited to, the following: (1) Cₑₒᵢ—concentration at end-of-infusion (IV); (2) Cₘₐₓ peak concentration (SC); (3) C_{trough}—pre-infusion concentration; (4) tₑₒᵢ—time of end-of-infusion; (5) tₘₐₓ—time to peak concentration (SC); (6) AUC_{3w}—area under the plasma concentration-time curve after the first SC or IV dose; and (7) F—Absolute bioavailability after SC administration.

The impact of the immunogenicity of REGN2810 will be evaluated. Anti-drug antibody (ADA) variables include ADA status and titer as follows: (1) treatment emergent—defined as any positive post-dose ADA assay response when baseline results are negative; (2) treatment boosted—defined as any post-dose ADA response that is at least 9-fold over baseline titer levels; (3) titer values (titer value category); (4) low (titer<1,000); (5) moderate (1,000≤titer≤10,000); high (titer>10,000). The relationship between immunogenicity and PK of REGN2810 may be assessed, as appropriate.

### Study Design

Groups 1 and 2: After a screening period of up to 28 days, patients in each of Groups 1 and 2 receive up to twelve 56-day (8-week) treatment cycles for a total of up to 96 weeks of treatment. Each patient will receive 3 mg/kg REGN2810 IV on days 1, 15±3, 29±3, and 43±3 during each treatment cycle. Tumor assessments will be made at the end of each treatment cycle. Extensive safety evaluations will occur on day 1 of each cycle; routine safety evaluations will be conducted at each REGN2810 dosing visit

Group 3: This cohort enrolls patients with metastatic CSCC. Group 3 only begins enrollment after completion of enrollment to Group 1. The dose regimen is 350 mg IV every three weeks for up to 54 weeks.

Group 4: This cohort enrolls patients with advanced CSCC (metastatic [nodal or distant] or locally advanced). Group 4 only begins enrollment after completion of enrollment in Groups 1 through 3. The dose regimen is 600 mg IV every four weeks for up to 48 weeks.

Group 5: This cohort enrolls patients with advanced CSCC. The regimen is 438 mg SC, 1 dose, followed in 3 weeks by 350 mg IV every three weeks for up to 54 weeks of total treatment. The first 3 patients in Group 5 will be dosed on 3 separate days to monitor for injection site reactions (ISRs). If zero severe ISRs are observed in the first 3 patients, the rest of the cohort may enroll without restrictions on enrollment day (ie, more than 1 patient may initiate treatment on the same day). If one or greater severe ISR is observed, enrollment will pause. In that circumstance, resumption of enrollment in Group 5 may be permitted after review of all relevant data and consensus between the medical monitor and the designated Risk Management lead from the Pharmacovigilance & Risk Management department. The investigators involved in care of these patients may also be consulted.

Patients continue REGN2810 until the planned duration of therapy is complete (96 weeks for Groups 1 and 2, 54 weeks for Group 3 and 5, and 48 weeks for Group 4), or until disease progression, unacceptable toxicity, or withdrawal of consent. (Patients in Groups 1 or 2 who experience CR have option to discontinue treatment after 48 weeks).

The dosing regimens for Groups 1-5 are summarized in Tables 4-7.

Dose levels may be reduced according to Table 8.

Groups 1 through 5: Patients who do not experience progressive disease (PD) at the completion of the planned treatment period will enter follow-up for approximately 6 months. After completion of this follow-up period, patients will then enter an extended follow-up period for approximately 1 additional year with assessments every 4 months (Table 9).

TABLE 4
Dosing regimens for Groups 1 and 2

| | Cycle 1 | | | | | Cycles 2-12 | | | | | |
|---|---|---|---|---|---|---|---|---|---|---|---|
| | Day | Day | Day | Day | Day | Day | Day | Day | Day | Day | |
| Group | 1 | 15 ± 3 | 29 ± 3 | 43 ± 3 | 56 ± 3 | 1 | 15 ± 3 | 29 ± 3 | 43 ± 3 | 56 ± 3 | Dose |
| 1 | X | X | X | X | | X | X | X | X | | 3 mg/kg IV |
| 2 | X | X | X | X | | X | X | X | X | | 3 mg/kg IV |

TABLE 5
Dosing regimen for Group 3

| Cycle 1 | | | | Cycles 2-6 | | | | |
|---|---|---|---|---|---|---|---|---|
| Day | Day | Day | Day | Day | Day | Day | Day | |
| 1 | 22 ± 3 | 43 ± 3 | 63 ± 3 | 1 | 22 ± 3 | 43 ± 3 | 63 ± 3 | Dose |
| X | X | X | | X | X | X | | 350 mg IV |

TABLE 6
Dosing regimen for Group 4

| Cycle 1 | | | Cycles 2-6 | | | |
|---|---|---|---|---|---|---|
| Day | Day | Day | Day | Day | Day | |
| 1 | 29 ± 3 | 56 ± 3 | 1 | 29 ± 3 | 56 ± 3 | Dose |
| X | X | | X | X | | 350 mg IV |

TABLE 7
Dosing regimen for Group 5

| Cycle 1 | | | Cycles 2-6 | | | |
|---|---|---|---|---|---|---|
| Day | Day | Day | Day | Day | Day | |
| 1 | 22 ± 3 | 43 ± 3 | 1 | 22 ± 3 | 43 ± 3 | Dose |
| X | | | X | | | 438 mg SC |
| | X | X | | X | X | 350 mg IV |

TABLE 8
Dose reductions

| | | |
|---|---|---|
| Dose Level | Reduction Order | Dose |

Groups 1-2

| | | |
|---|---|---|
| Dose Level -1 | First dose | 1 mg/kg REGN2810 |
| | reduction | every 14 days |
| Dose Level -2 | Second dose | 0.3 mg/kg REGN2810 |
| | reduction | every 14 days |

Group 3

| | | |
|---|---|---|
| Dose Level -1 | First dose | 120 mg REGN2810 |
| | reduction | every 21 days |
| Dose Level -2 | Second dose | 60 mg REGN2810 |
| | reduction | every 21 days |

REGN2810 will be administered in an outpatient setting as an approximately 30-minute (±10 minutes) IV infusion. Longer infusion durations are acceptable if interruption is required. Group 1 and Group 2 patient doses will depend on individual body weight. The dose of REGN2810 must be adjusted each cycle for changes in body weight of ≥10%. Dose adjustments for changes in body weight of <10% will be at the discretion of the investigator.

REGN2810 is supplied as a liquid in sterile, single-use vials. Each vial of REGN2810 contains a concentration of 25 or 50 mg/mL (for IV infusion), or a concentration of 175 mg/mL (for SC injection).

### Procedures and Assessments

Screening procedures include: informed consent, genomics substudy informed consent (optional), inclusion/exclusion, medical/oncology history, demographics, complete physical examination and ECOG PS, 12-Lead ECG, vital signs and weight, height, brain MRI, viral serology, coagulation, urinalysis, laboratory tests (hematology, blood chemistry, serum HCG 572 Hour Predose, urinalysis, HBV, HCV, HIV), archived tissue for histological confirmation of CSCC, tumor biopsies for Group 2, CT/MRI and/or digital photography, and treatment assignment.

Efficacy procedures include: tumor imaging (computed tomography [CT] or magnetic resonance imaging [MRI]) and digital medical photography (for externally visible lesions) to measure tumor burden and to characterize the efficacy profile of study treatments using response criteria. Additional efficacy procedures include tumor measurements and tumor biopsies.

Safety procedures include: assessment of vital signs, physical examination, electrocardiogram (ECG), immune safety assays, immunoglobulin levels (IgG, IgM, IgE), adverse event (AE) monitoring, laboratory testing (including pregnancy testing for women of childbearing potential).

Immune safety assays include the measurement of rheumatoid factor (RF), TSH, C-reactive protein (CRP), and antinuclear antibody (ANA) titer and pattern. If, during the course of the study, a 4-fold or greater increase from baseline in RF or ANA or abnormal levels of TSH or CRP are observed, the following tests may also be performed: anti-DNA antibody, anti-Sjögren's syndrome A antigen (SSA) antibody (Ro), anti-Sjögren's syndrome B antigen (SSB) antibody (La), antithyroglobulin antibody, anti-LKM antibody, antiphospholipid antibody, anti-islet cell antibody, antineutrophil cytoplasm antibody, C3, C4, CH50.

Laboratory testing procedures include: hematology, blood chemistry, pregnancy test (women only), and urinanalyis.

PK/drug concentrations: PK samples will be collected for assessment of REGN2810 concentrations in serum.

Anti-drug antibody measurements: Serum samples will be collected for the assessments of Immunogenicity to REGN2810.

Genomics sub-study measurements include: sequence determination or single nucleotide polymorphism studies of candidate genes and surrounding genomic regions, whole-exome sequencing, whole-genome sequencing, and DNA copy number variation.

### Concomitant Medications and Procedures

Prohibited medications and procedures. While participating in this study, a patient may not receive any standard or investigational agent for treatment of a tumor other than REGN2810 as mono therapy. After communication with the sponsor, focal palliative treatment (e.g., radiation) would be allowed for local control of a tumor once a patient has completed 24 weeks of study treatment. Any other medication which is considered necessary for the patient's welfare, and which is not expected to interfere with the evaluation of the study drug, may be given at the discretion of the investigator.

Patients using immunosuppressive doses (>10 mg per day of prednisone or equivalent) of systemic corticosteroids other than for corticosteroid replacement will not be eligible for the study. It is recommended that patients do not receive systemic corticosteroids such as hydrocortisone, prednisone, prednisolone (Solu-Medrol®) or dexamethasone (Decadron®) at any time throughout the study except in the case of a life-threatening emergency and/or to treat an irAE. Physiologic replacement doses of systemic corticosteroids are permitted, even if >10 mg/day prednisone equivalents. A brief course of corticosteroids for prophylaxis (e.g., contrast dye allergy) or for treatment of non-autoimmune conditions (e.g., delayed-type hypersensitivity reaction caused by contact allergen) is permitted. Bisphosphonates and denosumab are not prohibited

Surgery: For patients with locally advanced target lesions that are considered unresectable at baseline, but are subsequently deemed resectable during the course of the study due to tumor response to REGN2810, curative intent surgery may be allowed but must be discussed with the medical monitor prior to any surgical procedure. (This statement does not apply to patients in emergency life-threatening situations that require immediate surgery). Patients with inoperable CSCC at baseline who are rendered operable with clear margins will be deemed to have experienced PR.

If during the course of the study a patient develops new cutaneous lesions that are suspected to be a non-melanoma skin cancer other than CSCC (e.g., BCC), removal of the lesion and continued treatment on study may be allowed after discussion with the medical monitor.

Radiation therapy: Radiation therapy is not part of the study regimen. Patients for whom radiation therapy is planned are not eligible. If during the course of the study, a patient develops a symptomatic lesion for which palliative radiation therapy is deemed appropriate by the investigator, this will be deemed PD and generally the patient would be removed from study. Palliative radiation therapy may be allowed in certain circumstances in patients who have been on study for at least 24 weeks. Such cases must be discussed with the medical monitor prior to any radiation therapy if the investigator feels that restarting REGN2810 after radiation is in the best interest of the patient. The patient will be deemed to have experienced disease progression if radiation therapy is instituted, but will be followed for OS.

### WHO Criteria for Externally Visible Tumor(s) Require Bi-Dimensional Measurements

Clinical response criteria for externally visible tumor(s) require bi-dimensional measurements according to WHO criteria, and are as follows:

Complete response of externally visible disease (vCR): all target lesion(s) and non-target lesion(s) no longer visible, maintained for at least 4 weeks. Documentation of vCR requires confirmation by biopsies of site(s) of externally visible target lesion(s) with histologic confirmation of no residual malignancy, per central pathology review (Appendix 5). In the absence of such histologic confirmation, a patient cannot be deemed to have experienced vCR and the best response would be partial response.

Partial response of externally visible disease (vPR): decrease of 50% (WHO criteria) or greater in the sum the products of perpendicular longest dimensions of target lesion(s), maintained for at least 4 weeks.

Stable externally visible disease (vSD): not meeting criteria for vCR, vPR, or progressive disease.

Progression of visible disease (vPD): increase of 25% (WHO criteria) in the sum of the products of perpendicular longest dimensions of target lesion(s). In rare cases, unequivocal progression of a non-target lesion may be accepted as vPD.

### Results

It is expected that administration of REGN2810 will lead to enhanced tumor regression and improved disease control. Further, it is expected that administration of REGN2810 will lead to increased safety in patients with CSCC, with a reduced or zero incidence of adverse events and/or toxicity. For patients that do not have metastatic CSCC, it is expected that the likelihood of developing metastatic CSCC will be reduced.

193 patients (pts) were enrolled (Gp 1, n=59; Gp 2, n=78; Gp 3, n=56). 128 pts had received no prior anti-cancer systemic therapy, 65 pts were previously treated. As of Oct. 11, 2019 (data cut-off), median duration of follow-up was 15.7 months (range: 0.6-36.1) among all pts; 18.5 months (range: 1.1-36.1) for Gp 1, 15.5 months (range: 0.8-35.0) for Gp 2, and 17.3 months (range: 0.6-26.3) for Gp 3. ORR per INV was 54.4% (95% CI: 47.1-61.6) for all pts; 50.8% (95% CI: 37.5-64.1) for Gp 1, 56.4% (95% CI: 44.7-67.6) for Gp 2, and 55.4% (95% CI: 41.5-68.7) for Gp 3. ORR per INV was 57.8% (95% CI: 48.8-66.5) among treatment-naïve pts and 47.7% (95% CI: 35.1-60.5) among previously treated pts. Median duration of response (DOR) has not been reached (observed DOR range: 1.8-34.2 months). In responding pts, estimated DOR at 24 months was 76.0% (95% CI: 64.1-84.4). Median OS has not been reached. Estimated OS at 24 months was 73.3% (95% CI: 66.1-79.2). The most common treatment-emergent adverse events (TEAEs) by any grade were fatigue (34.7%), diarrhea (27.5%), and nausea (23.8%). The most common grade≥3 TEAEs were hypertension (4.7%) and anemia and cellulitis (each 4.1%).

For patients with advanced CSCC, cemiplimab achieves DOR and survival superior to what has been reported with other agents.

We claim:

1. A method of treating or inhibiting the growth of a tumor, comprising:
(a) selecting a patient with a skin cancer, wherein the patient has completed surgery and/or radiation therapy to treat the skin cancer; and
(b) subsequently administering to the patient an adjuvant treatment comprising a therapeutically effective amount of a programmed death 1 (PD-1) inhibitor.

2. The method of claim 1, wherein the patient has completed surgery and optionally, post-surgery radiation therapy.

3. The method according to claim 1 or 2, wherein the skin cancer is cutaneous squamous cell carcinoma (CSCC), basal cell carcinoma (BCC), Merkel cell carcinoma, or melanoma.

4. The method according to any one of claims 1-3, wherein the skin cancer is CSCC.

5. The method according to any one of claims 1-4, wherein the patient is at high risk of CSCC recurrence or has suffered at least one incident of recurrence.

6. The method according to claim 5, wherein the patient has at least one of the following high-risk features:
(a) nodal disease with extracapsular extension and at least 1 node>20 mm;
(b) in-transit metastases (ITM);
(c) T4 lesion;
(d) perineural invasion (PNI); and
(e) recurrent CSCC plus at least one of the following additional features:
(i) ≥N2b disease associated with a recurrent lesion;
(ii) nominal≥T3; and
(iii) ≥20 mm diameter of recurrent lesion.

7. The method according to any one of claims 1-6, wherein the therapeutically effective amount comprises 5 mg to 500 mg of the PD-1 inhibitor.

8. The method according to any one of claims 1-6, wherein the therapeutically effective amount comprises 350 mg of the PD-1 inhibitor.

9. The method according to any one of claims 1-8, wherein the PD-1 inhibitor is administered as one or more doses, wherein each dose is administered 2 to 12 weeks, preferably 3 weeks after the immediately preceding dose.

10. The method of claim 9, wherein each dose comprises 5 mg to 500 mg, preferably 350 mg of the PD-1 inhibitor.

11. The method according to any one of claims 1-10, wherein the PD-1 inhibitor is administered intravenously.

12. The method according to any one of claims 1-11, wherein step (b) occurs 2 to 6 weeks after completion of the radiation therapy.

13. The method according to any one of claims 1-12, wherein administration of the PD-1 inhibitor leads to reduced risk of subsequent skin cancer recurrence or zero incidence of subsequent skin cancer recurrence.

14. The method according to any one of claims 1-12, wherein administration of the PD-1 inhibitor leads to at least about 10% lower incidence of subsequent skin cancer recurrence as compared to a patient after completion of surgery and radiation therapy without adjuvant skin cancer treatment.

15. The method according to any one of claims 1-14, further comprising administering an additional therapeutic agent selected from a chemotherapeutic, a corticosteroid, an anti-inflammatory drug, and/or combinations thereof.

16. The method according to any one of claims 1-15, wherein the PD-1 inhibitor is selected from the group consisting of an anti-PD-1 antibody or antigen-binding fragment thereof, an anti-PD-L1 antibody or antigen-binding fragment thereof, and an anti-PD-L2 antibody or antigen-binding fragment thereof.

17. The method according to any one of claims 1-16, wherein the PD-1 inhibitor is an anti-PD-1 antibody or antigen-binding fragment thereof that comprises three complementarity determining regions (CDRs) (HCDR1, HCDR2, and HCDR3) of a heavy chain variable region (HCVR) comprising the amino acid sequence of SEQ ID NO: 1 and three CDRs (LCDR1, LCDR2, and LCDR3) of a light chain variable region (LCVR) comprising the amino acid sequence of SEQ ID NO: 2.

18. The method according to claim 17, wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8.

19. The method according to claim 17 or 18, wherein the anti-PD-1 antibody or antigen-binding fragment thereof comprises a HCVR/LCVR sequence pair of SEQ ID NOs: 1/2.

20. The method according to any one of claims 17-19, wherein the anti-PD-1 antibody comprises a heavy chain and a light chain, wherein the heavy chain has an amino acid sequence of SEQ ID NO: 9.

21. The method according to any one of claims 17-19, wherein the anti-PD-1 antibody comprises a heavy chain and a light chain, wherein the light chain has an amino acid sequence of SEQ ID NO: 10.

22. The method according to any one of claims 17-19, wherein the anti-PD-1 antibody comprises a heavy chain and a light chain, wherein the heavy chain has an amino acid sequence of SEQ ID NO: 9 and the light chain has an amino acid sequence of SEQ ID NO: 10.

23. The method according to any one of claims 1-16, wherein the PD-1 inhibitor is cemiplimab or a bioequivalent thereof.

24. The method according to any one of claims 1-16, wherein the PD-1 inhibitor is an anti-PD-1 antibody selected from the group consisting of cemiplimab, nivolumab, pembrolizumab, pidilizumab, MEDI0608, BI 754091, PF-06801591, spartalizumab, camrelizumab, JNJ-63723283, and MCLA-134.

25. The method according to any one of claims 1-16, wherein the PD-1 inhibitor is an anti-PD-L1 antibody selected from the group consisting of H1H8314N, avelumab, atezolizumab, durvalumab, MDX-1105, LY3300054, FAZ053, STI-1014, CX-072, KN035, and CK-301.

26. A pharmaceutical composition comprising a therapeutically effective amount of a programmed death 1 (PD-1) inhibitor for use in an adjuvant treatment of skin cancer after completion of surgery and post-surgery radiation.

27. The pharmaceutical composition of claim 26, wherein the PD-1 inhibitor is an anti-PD-1 antibody or antigen-binding fragment thereof comprising three complementarity determining regions (CDRs) (HCDR1, HCDR2, and HCDR3) of a heavy chain variable region (HCVR) comprising the amino acid sequence of SEQ ID NO: 1 and three CDRs (LCDR1, LCDR2, and LCDR3) of a light chain variable region (LCVR) comprising the amino acid sequence of SEQ ID NO: 2.

28. The pharmaceutical composition of claim 27, wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8.

29. The pharmaceutical composition of claim 28 wherein the anti-PD-1 antibody or antigen-binding fragment thereof comprises a HCVR/LCVR sequence pair of SEQ ID NOs: 1/2.

30. The pharmaceutical composition according to any one of claims 26-29, comprising 5 mg to 500 mg of the PD-1 inhibitor.

31. The pharmaceutical composition according to any one of claims 26-30, comprising 350 mg of the PD-1 inhibitor.

32. The pharmaceutical composition according to any one of claims 26-31, wherein the skin cancer is CSCC.

33. A method of treating or inhibiting the growth of a tumor, comprising:
(a) selecting a patient with a skin cancer for which surgical removal is planned; and
(b) prior to the surgical removal, administering to the patient a neoadjuvant treatment comprising a therapeutically effective amount of a programmed death 1 (PD-1) inhibitor.

34. The method according to claim 33, wherein the skin cancer is cutaneous squamous cell carcinoma (CSCC), basal cell carcinoma (BCC), Merkel cell carcinoma, or melanoma.

35. The method according to claim 33 or 34, wherein the skin cancer is CSCC.

36. The method according to any one of claims 33-35, wherein the patient is at high risk of CSCC recurrence.

37. The method according to claim 36, wherein the patient has at least one of the following high-risk features:
(a) nodal disease with extracapsular extension and at least 1 node>20 mm;
(b) in-transit metastases (ITM);
(c) T4 lesion;
(d) perineural invasion (PNI); and
(e) recurrent CSCC plus at least one of the following additional features:
(i) ≥N2b disease associated with a recurrent lesion;
(ii) nominal≥T3; and
(iii) ≥20 mm diameter of recurrent lesion.

38. The method according to any one of claims 33-37, wherein the therapeutically effective amount comprises 5 mg to 500 mg of the PD-1 inhibitor administered as a neoadjuvant.

39. The method according to any one of claims 33-38, wherein the therapeutically effective amount comprises 350 mg of the PD-1 inhibitor administered as the neoadjuvant.

40. The method according to any one of claims 33-38, wherein one or more doses of the PD-1 inhibitor are administered as neoadjuvant treatment, wherein each dose is administered 2 to 12 weeks, preferably 3 weeks after the immediately preceding dose.

41. The method according to claim 40, wherein each dose comprises 5 mg to 500 mg, preferably 350 mg of the PD-1 inhibitor.

42. The method according to any one of claims 33-41, further comprising: (c) subsequent to the neoadjuvant treatment, surgically removing the skin cancer.

43. The method according to claim 42, further comprising administering to the patient an adjuvant treatment comprising a therapeutically effective amount of a PD-1 inhibitor after step (c), wherein the adjuvant PD-1 inhibitor may be the same as or different from the neoadjuvant PD-1 inhibitor.

44. The method according to claim 43, wherein the adjuvant treatment comprises administering 5 mg to 500 mg of the PD-1 inhibitor.

45. The method according to claim 43 or 44, wherein the adjuvant treatment comprises administering 350 mg of the PD-1 inhibitor.

46. The method according to any one of claims 33-42, wherein the PD-1 inhibitor is administered intravenously.

47. The method according to any one of claims 33-46, wherein administration of the PD-1 inhibitor leads to reduced risk of subsequent skin cancer recurrence or zero incidence of subsequent skin cancer recurrence.

48. The method according to any one of claims 33-46, wherein administration of the PD-1 inhibitor leads to at least about 10% lower incidence of subsequent skin cancer recurrence as compared to a patient after completion of surgery and radiation therapy without adjuvant skin cancer treatment.

49. The method according to any one of claims 33-46, further comprising administering an additional therapeutic agent selected from a chemotherapeutic, a corticosteroid, an anti-inflammatory drug, and/or combinations thereof.

50. The method according to any one of claims 33-49, wherein the PD-1 inhibitor is selected from the group consisting of an anti-PD-1 antibody or antigen-binding fragment thereof, an anti-PD-L1 antibody or antigen-binding fragment thereof, and an anti-PD-L2 antibody or antigen-binding fragment thereof.

51. The method according to any one of claims 33-50, wherein the PD-1 inhibitor is an anti-PD-1 antibody or antigen-binding fragment thereof that comprises three complementarity determining regions (CDRs) (HCDR1, HCDR2, and HCDR3) of a heavy chain variable region (HCVR) comprising the amino acid sequence of SEQ ID NO: 1 and three CDRs (LCDR1, LCDR2, and LCDR3) of a light chain variable region (LCVR) comprising the amino acid sequence of SEQ ID NO: 2.

52. The method according to claim 51, wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8.

53. The method according to claim 51 or 52, wherein the anti-PD-1 antibody or antigen-binding fragment thereof comprises a HCVR/LCVR sequence pair of SEQ ID NOs: 1/2.

54. The method according to any one of claims 51-53, wherein the anti-PD-1 antibody comprises a heavy chain and a light chain, wherein the heavy chain has an amino acid sequence of SEQ ID NO: 9.

55. The method according to any one of claims 51-53, wherein the anti-PD-1 antibody comprises a heavy chain and a light chain, wherein the light chain has an amino acid sequence of SEQ ID NO: 10.

56. The method according to any one of claims 51-53, wherein the anti-PD-1 antibody comprises a heavy chain and a light chain, wherein the heavy chain has an amino acid sequence of SEQ ID NO: 9 and the light chain has an amino acid sequence of SEQ ID NO: 10.

57. The method according to any one of claims 33-50, wherein the PD-1 inhibitor is cemiplimab or a bioequivalent thereof.

58. The method according to any one of claims 33-50, wherein the PD-1 inhibitor is an anti-PD-1 antibody selected from the group consisting of cemiplimab, nivolumab, pembrolizumab, pidilizumab, MED10608, BI 754048, PF-06371548, spartalizumab, camrelizumab, JNJ-63313240, and MCLA-134.

59. The method according to any one of claims 33-50, wherein the PD-1 inhibitor is an anti-PD-L1 antibody selected from the group consisting of H1H8314N, avelumab, atezolizumab, durvalumab, MDX-1105, LY3300054, FAZ053, STI-1014, CX-031, KN035, and CK-301.

60. A pharmaceutical composition comprising a therapeutically effective amount of a programmed death 1 (PD-1) inhibitor for use in a neoadjuvant treatment prior to planned surgery for treating skin cancer.

61. The pharmaceutical composition of claim 60, wherein the PD-1 inhibitor is an anti-PD-1 antibody or antigen-binding fragment thereof comprising three complementarity determining regions (CDRs) (HCDR1, HCDR2, and HCDR3) of a heavy chain variable region (HCVR) comprising the amino acid sequence of SEQ ID NO: 1 and three CDRs (LCDR1, LCDR2, and LCDR3) of a light chain variable region (LCVR) comprising the amino acid sequence of SEQ ID NO: 2.

62. The pharmaceutical composition of claim 61, wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of AAS; and LCDR3 has an amino acid sequence of SEQ ID NO: 8.

63. The pharmaceutical composition of claim 62, wherein the anti-PD-1 antibody or antigen-binding fragment thereof comprises a HCVR/LCVR sequence pair of SEQ ID NOs: 1/2.

64. The pharmaceutical composition according to any one of claims 60-63, comprising 5 mg to 500 mg of the PD-1 inhibitor.

65. The pharmaceutical composition according to any one of claims 60-63, comprising 350 mg of the PD-1 inhibitor.

66. The pharmaceutical composition according to any one of claims 60-65, wherein the skin cancer is CSCC.
